(19) United States
(12) Patent Application Publication
(10) Pub. No.: US 2010/0202998 A1
(43) Pub. Date: Aug. 12, 2010

(54) USE OF AT LEAST ONE LYSINE-DERIVED COMPOUND FOR THE CONDITIONING OF KERATINOUS FIBERS, COSMETIC COMPOSITION COMPRISING IT AND METHOD FOR CONDITIONING THE FIBERS

(76) Inventors: Laure RAMOS-STANBURY, Sceaux (FR); Lourdes Peres Munoz, Barcelona (ES); Maria Roase Infante Martinez-Pardo, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/606,380

(22) Filed: Oct. 27, 2009

Related U.S. Application Data
(60) Provisional application No. 61/121,279, filed on Dec. 10, 2008.

(30) Foreign Application Priority Data
Oct. 27, 2008 (FR) 0857287

(51) Int. Cl. A61K 8/44; A61K 8/72; A61K 8/89; A61K 8/73; A61K 8/84; A61Q 5/12; A61Q 5/02

(52) U.S. Cl. 424/70.12; 424/70.1; 424/70.11; 424/70.13; 424/70.15; 424/70.17; 424/70.21; 424/70.22; 424/70.27; 424/70.28

(57) ABSTRACT

The present disclosure relates to a method for the conditioning of keratinous fibers comprising applying to dry or wet fibers, a cosmetic composition comprising at least one entity chosen from lysine-derived compounds of formula (I), and the salts and/or solvates thereof. The disclosure also relates to a cosmetic composition for conditioning keratinous fibers comprising, in a cosmetically acceptable medium, at least one entity chosen from lysine-derived compounds of formula (I) and the salts and/or solvates thereof and at least one cosmetic adjuvant. The disclosure also relates to a mixture of at least one entity chosen from lysine-derived compounds of formula (I) and the salts and/or solvents thereof.

This application claims benefit of U.S. Provisional Application No. 61/121,279, filed Dec. 10, 2008, the contents of which are incorporated herein by reference. This application also claims benefit of priority under 35 U.S.C. §119 to French Patent Application No. FR 0857287, filed Oct. 27, 2008, the contents of which are also incorporated herein by reference.

The present disclosure relates to the use of at least one entity chosen from specific lysine-derived compounds and the salts and/or solvates thereof for the conditioning of keratinous fibers, for example human keratinous fibers, such as the hair. The disclosure also relates to a method for conditioning keratinous fibers employing a composition comprising these compounds.

Another aspect of the present disclosure is likewise a cosmetic composition comprising, in a cosmetically acceptable medium, at least one entity chosen from specific lysine-derived compounds and the salts and/or solvates thereof, and a method of using of this composition for the conditioning of fibers.

The hair can be generally damaged and embrittled by the action of external atmospheric agents, such as light and bad weather, and by mechanical or chemical treatments, such as brushing, combing, dyeing, bleaching, perming and/or straightening.

For example, in the case of a perming or straightening, it has been observed that the quality of the hair may often be detrimentally affected. The essential consequences of this detrimental change may be a deterioration in its cosmetic properties, such as gloss, and a deterioration in its mechanical properties, for example a deterioration in its strength due to swelling of the individual hairs during rinsing between the stage of application of a composition comprising a reducing agent (reduction stage) and the stage of application of a composition comprising an oxidizing agent (oxidation stage). This deterioration can also be reflected by an increase in the porosity of individual hairs.

The hair may thus be weakened and may become easily breakable during subsequent treatments, such as blow drying. It also results from this that such treatments generally may make the hair difficult to manage; for example, it may be difficult to disentangle or to style and heads of hair, even densely populated heads of hair, may find it difficult to retain a style which is attractive in appearance due to the fact that the hair lacks vigor and liveliness.

In order to overcome this, it is now common practice to apply haircare products comprising conditioning agents, for example cationic surfactants, in order to repair or limit the harmful or undesirable effects brought about by the various treatments or attacks to which the hair is more or less repeatedly subjected.

These haircare products can for example, be provided in the form of gels, creams, lotions, or conditioners.

However, these haircare products based on cationic surfactants generally do not confer entirely satisfactory cosmetic properties on the hair, such as in terms of disentangling, of smoothness, of suitability for combing, of styling and of softness.

Thus, there exists a real need to provide compounds for the conditioning of the hair in order to provide it with at least one satisfactory cosmetic property, such as in terms of disentangling, of smoothness, of suitability for combing, of softness and of feel, while exhibiting a low ecotoxicity and being easily biodegradable.

International Patent Application WO2006/056636 describes specific lysine-derived compounds, such as compounds of the methyl ester of N,N-acyloxypropyllysine and methyl ester of N,N-bis(N-acyloxypropyl)lysine type, their process of synthesis and their use generally in the food, pharmaceutical and cosmetic fields for their antimicrobial activity and their ability to self-aggregate.

It has now been discovered that the use of these specific lysine-derived compounds can make it possible to condition the hair, that is to say to confer, on the hair, at least one satisfactory cosmetic property in terms of disentangling, of smoothness, of suitability for combing, of softness and of feel, while exhibiting a low ecotoxicity and being easily biodegradable.

For example, it has been found that the use of these specific lysine-derived compounds can make it possible to improve at least one of the cosmetic properties which are conferred on the hair in comparison with the cationic surfactants conventionally used, such as regards its suitability for disentangling or for combing, and also in terms of softness and of smoothness.

Furthermore, in comparison with the surfactants conventionally used, these lysine-derived compounds exhibit a markedly reduced ecotoxicity and are more easily biodegradable.

Accordingly, one aspect of the present disclosure is thus the method for conditioning keratinous fibers, for example human keratinous fibers, such as the hair, comprising applying, to dry or wet fibers, a cosmetic composition comprising at least one entity chosen from lysine-derived compounds of following formula (I) and the salts and/or solvates thereof:

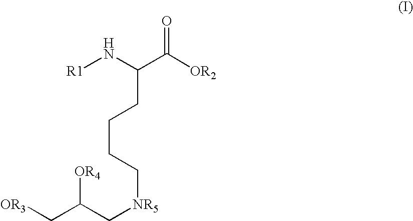

in which:

R₁ represents a hydrogen atom or a protective group;

R₂ represents a hydrogen atom, a linear C₁-C₆ alkyl radical or a branched C₃-C₆ alkyl radical;

R₃ and R₄ represent, independently of one another, a hydrogen atom, a saturated or unsaturated linear C₂-C₂₅ acyl group or a saturated or unsaturated branched C₄-C₂₅ acyl group;

R₅ represents a hydrogen atom or a CH₂CH(OR₆)CH₂OR₇ radical;

R₆ and R₇ represent, independently of one another, a hydrogen atom, a saturated or unsaturated linear C₃-C₂₅ acyl group or a saturated or unsaturated branched C₄-C₂₅ acyl group;

on the condition that R₁, R₂, R₃, R₄, R₅ and optionally R₆ and R₇, when they are present, cannot simultaneously represent a hydrogen atom;

optionally rinsing the cosmetic composition off after an optional leave-in time and/or after optionally drying the fibers.

These compounds can make it possible for example to improve the suitability for disentangling the hair and the contribution of softness in comparison with the cationic surfactants which are conventionally used in cosmetic compositions.

Another aspect of the present disclosure is a method for making for a cosmetic composition for keratinous fibers, for example human keratinous fibers, such as the hair, comprising combining at least one lysine-derived compounds of formula (I) as defined above and the salts and/or solvates thereof, and at least one adjuvant.

The present disclosure also relates to a cosmetic composition for conditioning keratinous fibers which can comprise, in a cosmetically acceptable medium, at least one entity chosen from the compounds of formula (I) as defined above; and at least one cosmetic adjuvant.

Another aspect of the present disclosure is at least one entity chosen from specific lysine-derived compounds corresponding to the formula (I) and the salts and/or solvates thereof and a cosmetic composition for the conditioning of keratinous fibers which comprises, in a cosmetically acceptable medium, such lysine-derived compounds.

However, other characteristics, aspects, subject-matters and possible benefits of the present disclosure will become even more clearly apparent on reading the description and examples which follow.

Protective group is understood to mean, within the meaning of the present disclosure, a functional group introduced into the molecule in order to protect the amine group from side reactions.

For example, the protective group can be chosen from a linear or branched C₂-C₆ acyl group, such as an acetyl group; a linear or branched C₁-C₆ alkyl group, such as a methyl or ethyl group; a C₁-C₆ carboxyalkoxy group; a (C₁-C₆)alkoxycarbonyl group; a linear or branched C₁-C₆ alkyl group interrupted by at least one nitrogen atom and/or a carboxyl group; a C₂-C₆ sulphonyl group; and an aryl group.

For example, the protective group can be a linear or branched C₂-C₆ acyl group, such as an acetyl group.

According to at least one embodiment, R₁ represents a hydrogen atom; R₂ represents a linear C₁-C₄ alkyl radical, such as a methyl or ethyl group, or a branched C₃-C₄ alkyl radical, such as a tert-butyl group; R₃ and R₄ represent, independently of one another, a hydrogen atom or a saturated or unsaturated and linear or branched C₁₂-C₂₂, such as C₁₆-C₂₂, acyl group; R₅ represents a hydrogen atom or a CH₂CH(OR₆)CH₂OR₇ group; R₆ and R₇ represent, independently of one another, a hydrogen atom or a saturated or unsaturated and linear or branched C₁₂-C₂₂, for example C₁₆-C₂₂, acyl group; and on the condition that R₁, R₂, R₃, R₄, R₅ and optionally R₆ and R₇ cannot simultaneously represent a hydrogen atom.

According to another embodiment, R₁ represents a hydrogen atom; R₂ represents a branched C₃-C₄ alkyl radical, such as a tert-butyl group; R₃ represents a saturated or unsaturated and linear or branched C₁₂-C₂₂, such as C₁₆-C₂₂, acyl group; R₄ represents a hydrogen atom; R₅ represents a CH₂CH(OR₆)CH₂OR₇ group; R₆ represents a hydrogen atom or a saturated or unsaturated and linear or branched C₁₂-C₂₂, such as C₁₆-C₂₂, acyl group; and R₇ represents a saturated or unsaturated and linear or branched C₁₂-C₂₂, such as C₁₆-C₂₂, acyl group.

According to another embodiment, R₁ represents a hydrogen atom; R₂ represents a linear C₁-C₄ alkyl radical, such as a methyl or ethyl group; R₃ represents a saturated or unsaturated and linear or branched C₁₂-C₂₂, such as C₁₆-C₂₂, acyl group; R₄ represents a hydrogen atom; R₅ represents a CH₂CH(OR₆)CH₂OR₇ group; R₆ represents a hydrogen atom or a saturated or unsaturated and linear or branched C₁₂-C₂₂, such as C₁₆-C₂₂, acyl group; and R₇ represents a saturated or unsaturated and linear or branched C₁₂-C₂₂, such as C₁₆-C₂₂, acyl group.

Thus, the at least one entity chosen from lysine-derived compound of formula (I) and the salts and/or solvates thereof could be an ester which is mono-, di-, tri- or tetrasubstituted.

In accordance with at least one embodiment of the disclosure, the at least one entity chosen from lysine-derived compounds of formula (I) can be chosen from:

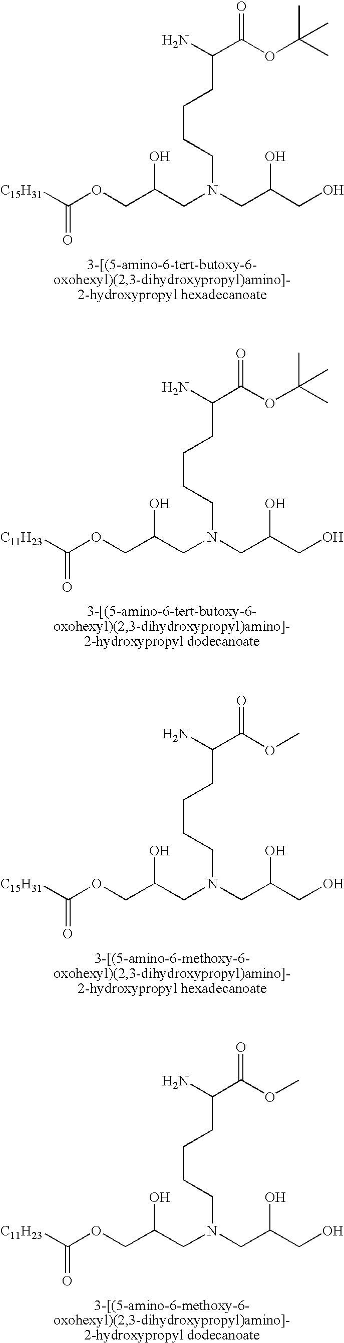

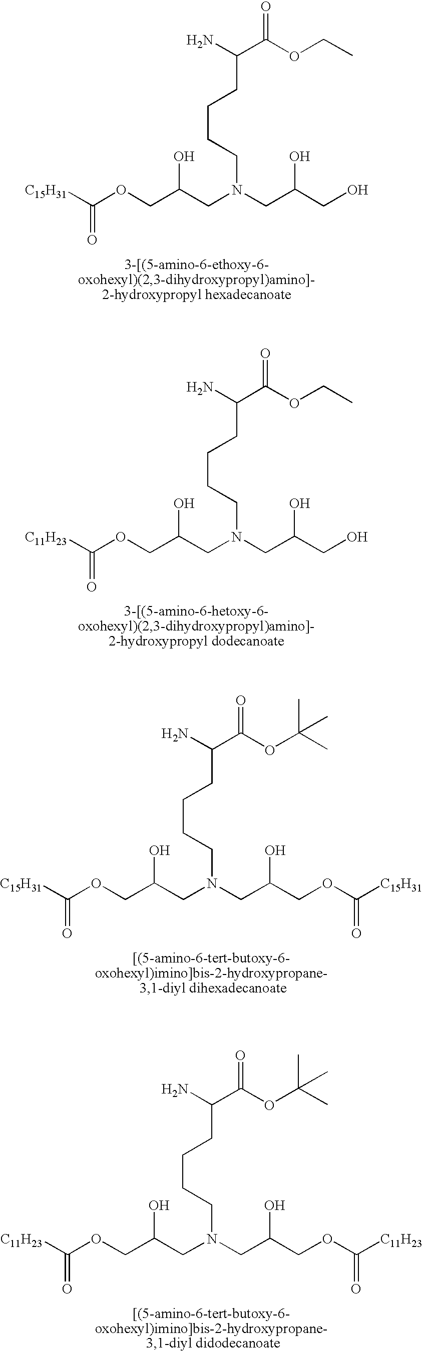

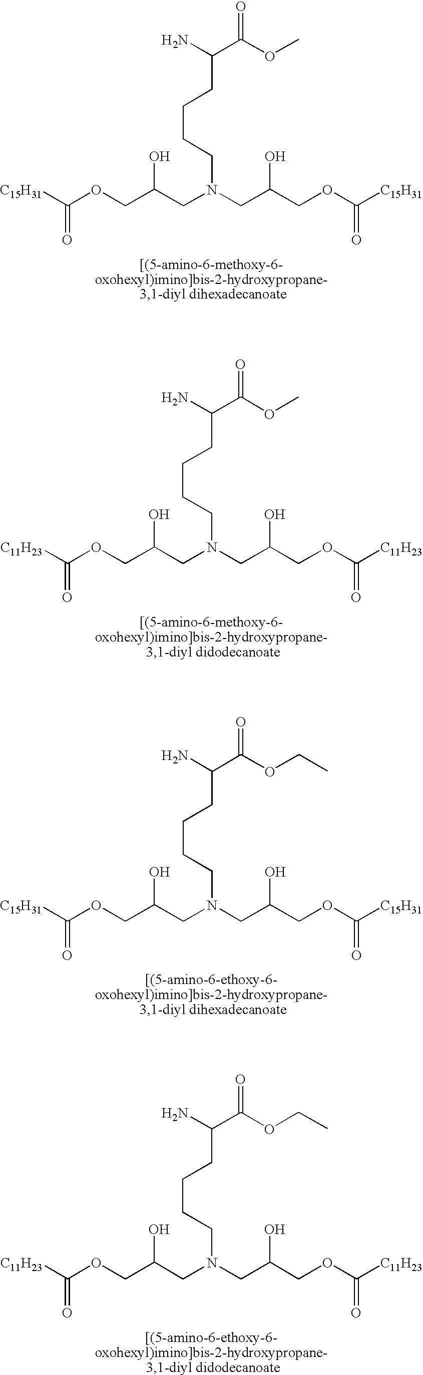

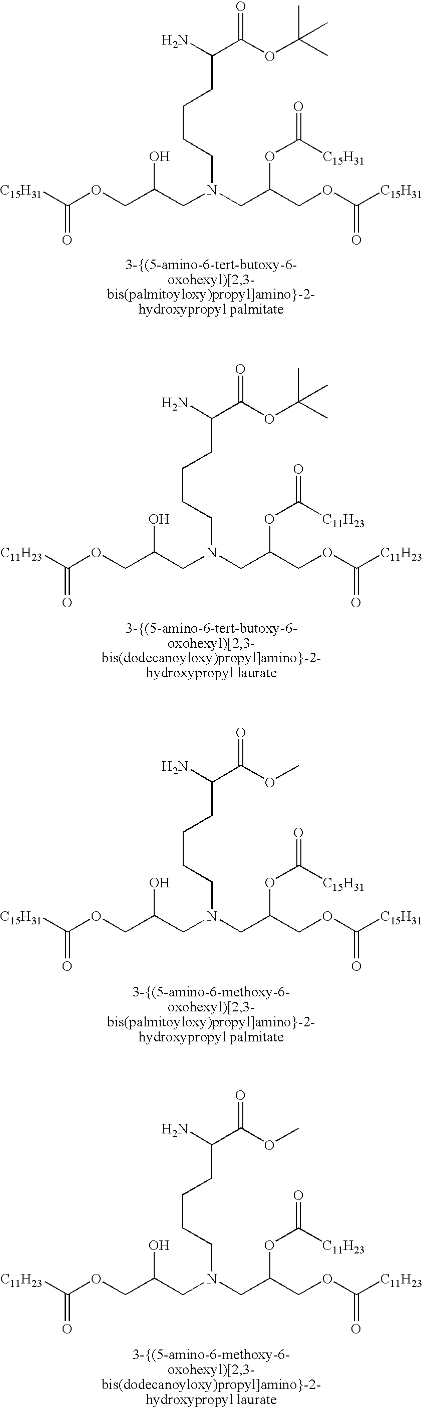

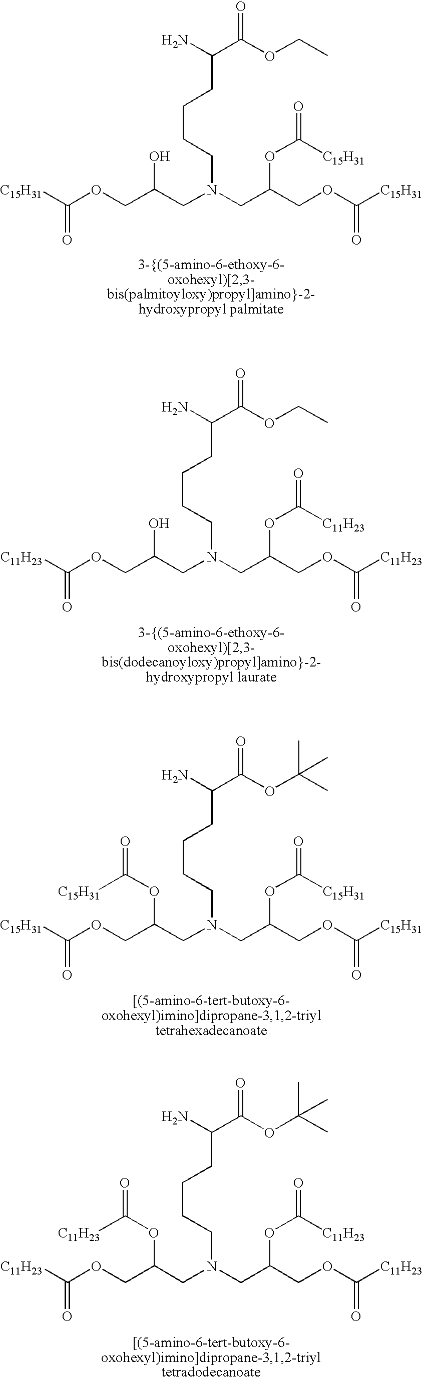

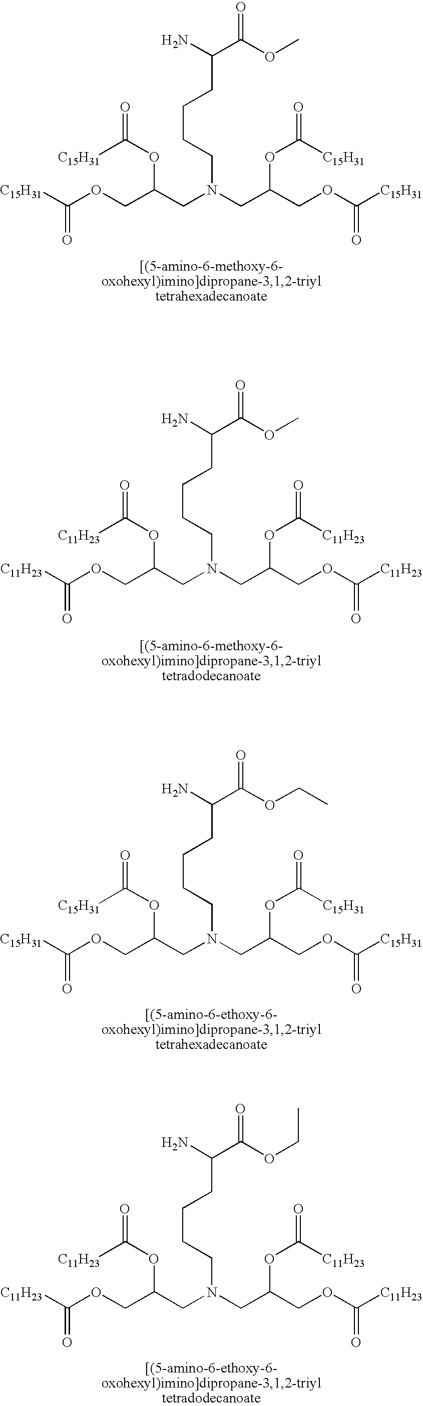

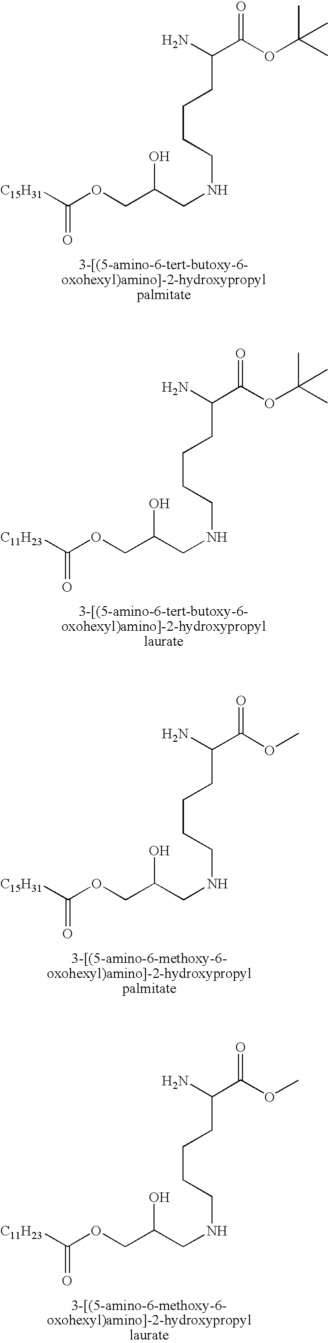

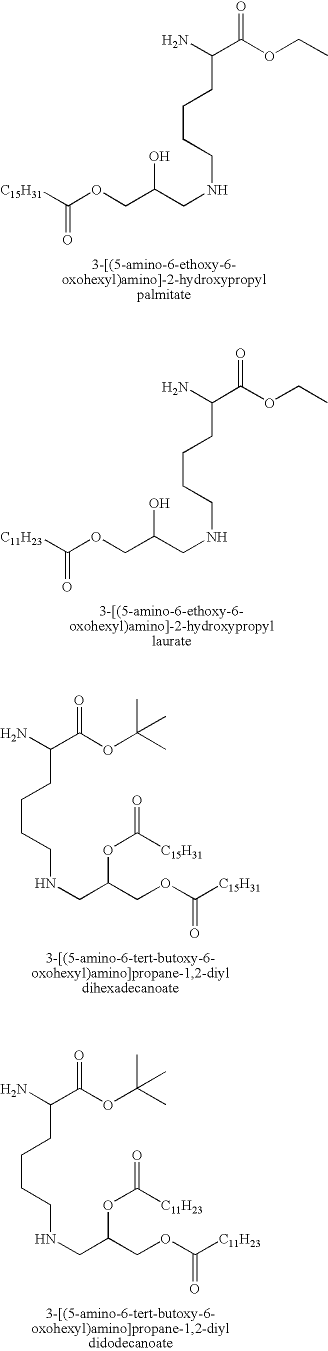

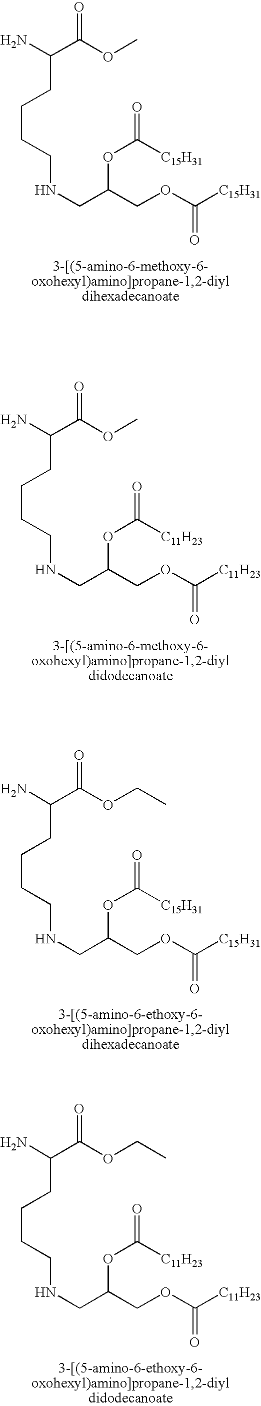

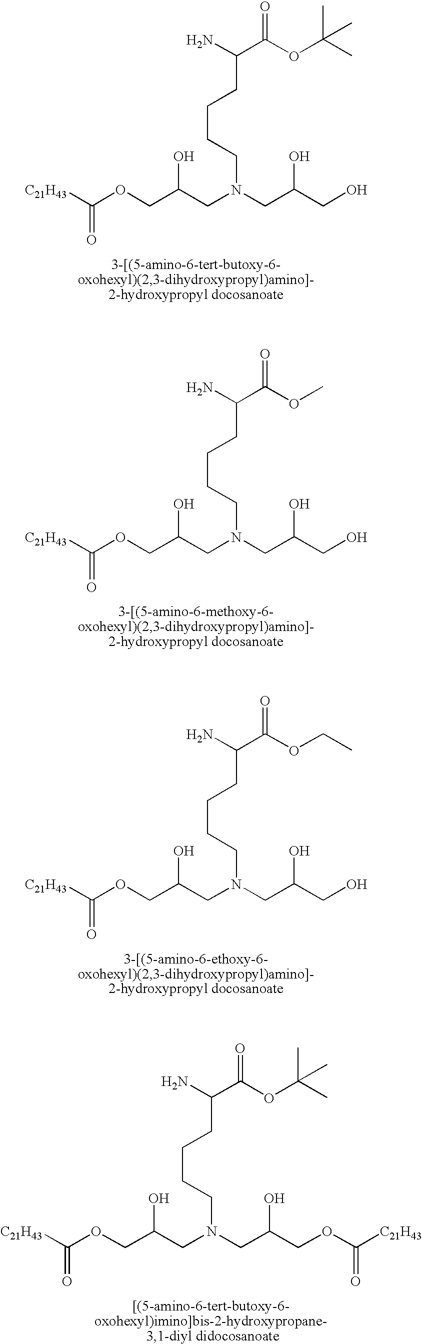

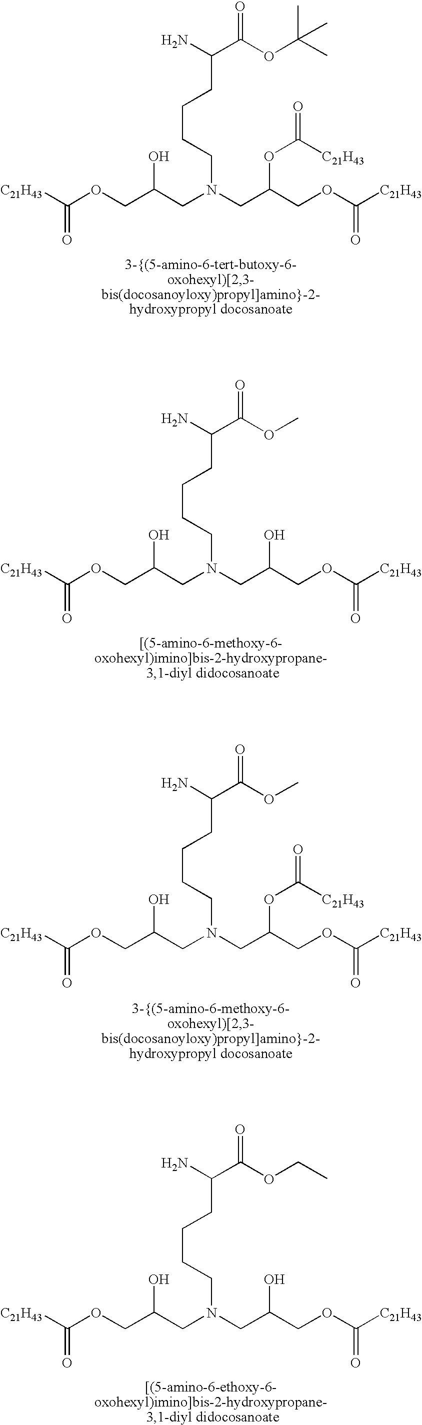

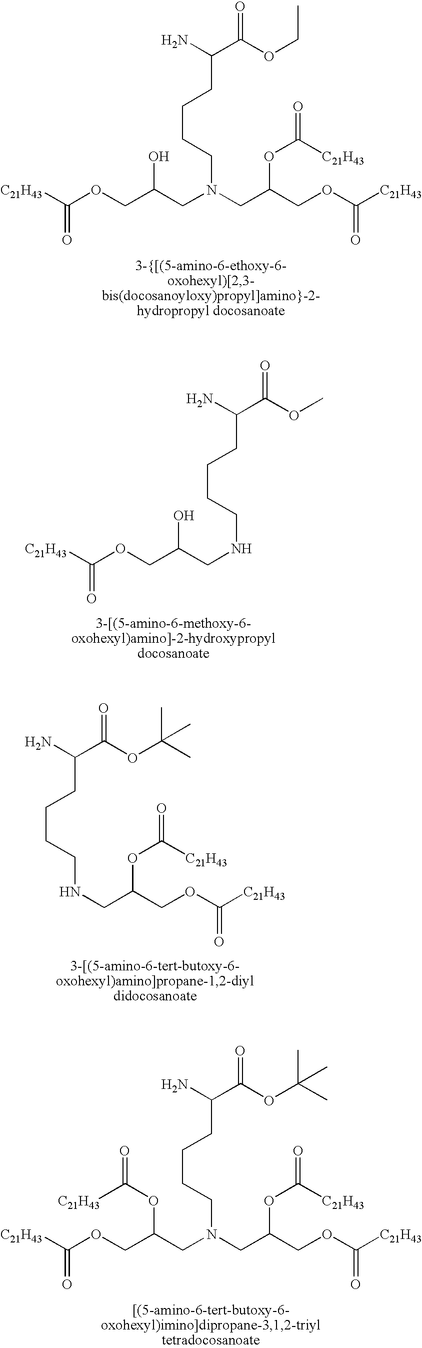

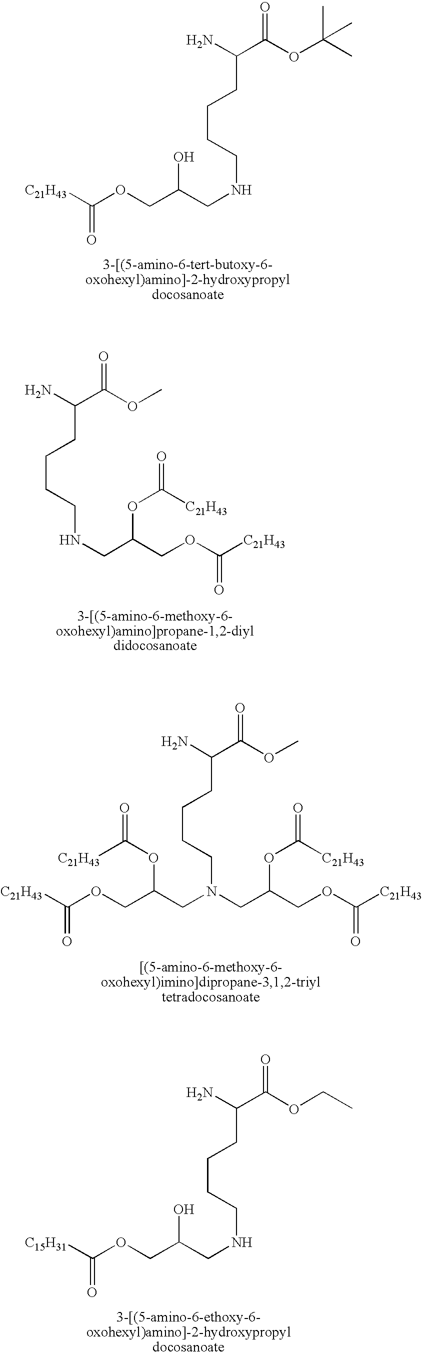

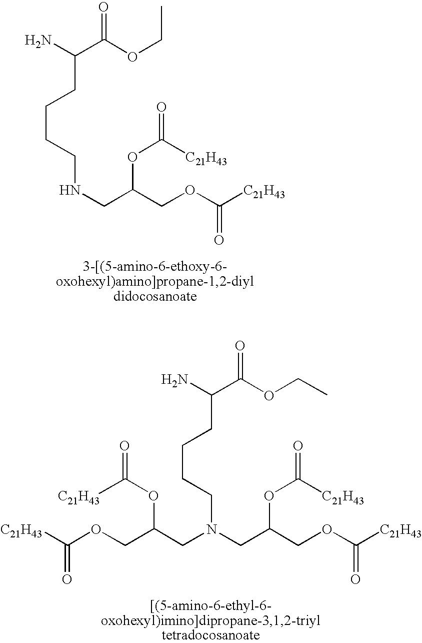

and the salts and/or solvates thereof.

For example, in at least one embodiment, the present disclosure relates to the use of a mixture of several entities chosen from lysine-derived compound of formula (I) and the salts and/or solvates thereof, such as of mono-, di-, tri- and tetrasubstituted esters.

Salt is understood to mean, within the meaning of the present disclosure, the organic or inorganic salts of the at least one entity chosen from specific lysine-derived compounds of formula (I).

Mention may be made, among the inorganic salts, of hydrohalides (for example hydrochlorides), carbonates, hydrogencarbonates, sulphates, hydrogenphosphates, or phosphates.

The organic salts which can be used are, for example, the salts of organic acids, such as citrates, lactates, glycolates, gluconates, acetates, propionates, fumarates, oxalates, and tartrates.

Solvate is understood to mean, within the meaning of the present disclosure, a stoichiometric mixture of the at least one entity chosen from lysine-derived compound of formula (I) and the salts with at least one molecule of water or of organic solvent.

Mention may be made, as possible solvates of the at least one entity chosen from lysine-derived compounds of formula (I) and the salts, of hydrates, alcoholates, and hydroalcoholates.

The synthesis of these compounds is known and is, for example, described in International Patent Application WO2006/056636.

As discussed above, the present disclosure relates to a method for caring for keratinous fibers comprising applying, to the dry or wet fibers, a cosmetic composition comprising at least one entity chosen from specific lysine-derived compound of formula (I) and the salts and/or solvates thereof and optionally rinsing off after an optional leave-in time or after an optional drying.

For example, the application of the cosmetic composition comprising at least one entity chosen from the lysine-derived compounds of formula (I) and the salts and/or solvates thereof may possibly not be followed by rinsing.

The disclosure for example relates to a composition for conditioning keratinous fibers, for example human keratinous fibers, such as the hair, comprising, in a cosmetically acceptable medium, at least one entity chosen from lysine-derived compounds of formula (I) and the salts and/or solvates thereof as defined above and at least one cosmetic adjuvant.

The at least one entity chosen from lysine-derived compounds of formula (I) and the salts and/or solvates thereof can be present in a content by weight ranging from 0.01 to 50% by weight, such as in a content ranging from 0.1 to 30% by weight, further such as from 0.5 to 25% by weight, even further such as from 1 to 20% by weight, further still such as from 1.5 to 10% by weight, with respect to the total weight of the composition.

The at least one cosmetic adjuvant present in the cosmetic composition is chosen from surface-active agents other than the compounds of formula (I), polymers, silicones, ceramides, and pseudoceramides, vitamins and provitamins, water-soluble and fat-soluble sunscreens, solid compounds, such as pigments, pearlescent or opacifying agents, sequestering agents, plasticizing agents, solubilizing agents, acidifying agents, basifying agents, neutralizing agents, inorganic and organic thickening agents, antifoaming agents, fragrances, corrosion inhibitors, fatty substances, such as vegetable, animal, mineral and synthetic oils, reducing agents, oxidizing agents, esters, emollients, antioxidants, hydroxy acids, solvents, penetrating agents, buffers, dispersing agents, preservatives and pH-modifying agents.

The at least one cosmetic adjuvant may be chosen, for example, from anionic surfactants, cationic surfactants other than the compounds of formula (I), amphoteric surfactants, and nonionic surfactants, cationic polymers, anionic polymers, amphoteric polymers, zwitterionic polymers, and nonionic polymers, and silicones.

According to another embodiment, the at least one cosmetic adjuvant may be a surfactant chosen from anionic surfactants, cationic surfactants, amphoteric surfactants, and nonionic surfactants.

The anionic surfactants which can be used in the compositions of the disclosure are chosen for example from the salts, such as the alkali metal salts, further such as the sodium salts, the ammonium salts, the amine salts, the aminoalcohol salts or the alkaline earth metal salts, for example magnesium salts, of the following types: alkyl sulphates, alkyl ether sulphates, alkylamido ether sulphates, alkylaryl polyether sulphates, monoglyceride sulphates, alkylsulphonates, alkylamidesulphonates, alkylarylsulphonates, olefinsulphonates, paraffinsulphonates, alkyl sulphosuccinates, alkyl ether sulphosuccinates, alkylamidesulphosuccinates, alkyl suiphoacetates, acylsarcosinates and acylgiutamates, the alkyl and acyl groups of all these compounds comprising from 6 to 24 carbon atoms and the aryl group such as denoting a phenyl or benzyl group.

Use may also be made of C₆₋₂₄ alkyl monoesters of polyglycosidedicarboxylic acids, such as alkyl glucoside citrates, alkyl polyglycoside tartrates, and alkyl polyglycoside sulphosuccinates, alkyl sulphosuccinamates, acylisothionates, and N-acyltaurates, the alkyl or acyl group of all these compounds comprising from 12 to 20 carbon atoms.

Another group of anionic surface-active agents which can be used in the compositions of the present disclosure is that of the acyllactylates, the acyl group of which comprises from 8 to 20 carbon atoms.

In addition, mention may also be made of alkyl-D-galactosideuronic acids and their salts and also C₆₋₂₄ alkyl polyoxyalkylenated ether carboxylic acids, (C₆₋₂₄ alkyl)(C₆₋₂₄ aryl)polyoxyalkylenated ether carboxylic acids, (C₆₋₂₄ alkyl)amido polyoxyalkylenated ether carboxylic acids and their salts, for example those comprising from 2 to 50 ethylene oxide units, and their mixtures.

For example, use may be made of alkyl sulphates, alkyl ether sulphates, and their mixtures, such as in the form of alkali metal, alkaline earth metal, ammonium, amine or aminoalcohol salts.

Examples of nonionic surfactants which can be used in the compositions of the present disclosure are described, for example, in “Handbook of Surfactants” by M. R. Porter, published by Blackie & Son (Glasgow and London), 1991, pp 116-178. They are chosen for example from alcohols, α-diols and (C₁₋₂₀)alkyl phenols having a fatty chain comprising, for example, from 8 to 18 carbon atoms, it being possible for the number of ethylene oxide or propylene oxide groups to range for example from 2 to 50 and it being possible for the number of glycerol groups to range for example from 2 to 30.

Mention may also be made of condensates of ethylene oxide and of propylene oxide with fatty alcohols; polyethoxylated fatty amides such as having from 2 to 30 ethylene oxide units, polyglycerolated fatty amides comprising on average from 1 to 5 glycerol groups and such as from 1.5 to 4, ethoxylated sorbitan fatty acid esters having from 2 to 30 ethylene oxide units, sucrose fatty acid esters, polyethylene glycol fatty acid esters, (C₆₋₂₄ alkyl)polyglycosides, N—(C₆₋₂₄ alkyl)glucamine derivatives, or amine oxides, such as (C₁₀₋₁₄ alkyl)amine oxides or N—(C₁₀₋₁₄ acyl)aminopropylmorpholine oxides.

The amphoteric or zwitterionic surface-active agents which can be used in the present disclosure can for example be derivatives of secondary or tertiary aliphatic amines in which the aliphatic group may be a linear or branched chain comprising from 8 to 22 carbon atoms and comprising at least one anionic group, such as, for example, a carboxylate, sulphonate, sulphate, phosphate or phosphonate group. Mention may, for example, be made of (C₈₋₂₀)alkyl betaines, sulphobetaines, C₈₋₂₀ alkyl amido(C₆₋₈ alkyl)betaines or C₈₋₂₀ alkyl amido(C₆₋₈ alkyl)sulphobetaines.

Mention may be made, among amine derivatives, of the products sold under the name MIRANOL®, such as described in U.S. Pat. No. 2,528,378 and U.S. Pat. No. 2,781,354 and classified in the CTFA dictionary, 3^{rd} edition, 1982, under the names Amphocarboxyglycinate and Amphocarboxypropionate with the respective structures (A) and (B):

Rₐ—CONHCH₂CH₂—N(R_{b})(R_{c})(CH₂COO⁻) (A)

in which:

Rₐ represents an alkyl group derived from an Rₐ—COOH acid present in hydrolysed coconut oil or a heptyl, nonyl or undecyl group,

R_{b} represents a β-hydroxyethyl group, and

R_{c} represents a carboxymethyl group;

and

Rₐ′—CONHCH₂CH₂—N(B)(B′) (B)

in which:

B represents —CH₂CH₂OX′,

B′ represents —(CH₂)_{z}—Y′, with z=1 or 2,

X′ represents the —CH₂CH₂—COOH group or a hydrogen atom,

Y′ represents —COON or the —CH₂—CHOH—SO₃H group,

Rₐ′ represents an alkyl group of an acid Rₐ'—COOH present in hydrolysed linseed oil or coconut oil, an alkyl group, such as a C₁₋₇ alkyl group and its iso form, or an unsaturated C₁₇ group.

These compounds are classified in the CTFA dictionary, 5^{th} edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

Mention may be made, by way of example, of the cocoamphodiacetate sold by Rhodia under the trade name MIRANOL® C2M concentrate.

Use can be made for example, among the abovementioned amphoteric or zwitterionic surfactants, of C₈₋₂₀ alkyl betaines, C₈₋₂₀ alkyl amido(C₆₋₈ alkyl)betaines and their mixtures.

For example, in at least one embodiment, the composition according to the disclosure comprises at least one anionic surfactant and at least one amphoteric or zwitterionic surfactant.

Mention may, for example, be made, as examples of cationic surfactant, of optionally polyoxyalkylenated primary, secondary or tertiary fatty amine salts; quaternary ammonium salts, such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides having a cationic nature.

According to another embodiment, the cosmetic adjuvant may be chosen from polymers, such as cationic polymers, amphoteric polymers (such as zwitterionic) and nonionic polymers, for example cationic polymers, and combinations of these. “Polymer” as used here is understood to mean homopolymers and copolymers, the copolymers resulting from more than one type of monomer, for example from two, three or four types of different monomers or more.

Within the meaning of the present disclosure, the expression “cationic polymer” denotes any polymer comprising cationic groups and/or groups which can be ionized to give cationic groups.

The cationic polymers which can be used in accordance with the present disclosure can be chosen from all those already known per se as improving the cosmetic properties of the hair, namely, for example, those described in Patent Application EP-A-337 354 and in French Patents FR-2 270 846, 2 383 660, 2 598 611, 2 470 596 and 2 519 863.

For example, the cationic polymers can be chosen from those which comprise units comprising primary, secondary, tertiary and/or quaternary amine groups which can either form part of the main polymer chain or be carried by a side substituent directly connected to the main chain.

The cationic polymers used generally have a number-average molecular weight ranging from 500 to 5×10⁶ such as from 10³ to 3×10⁶.

Mention may, for example, be made, among cationic polymers, of the polymers of the polyamine, polyaminoamide and poly(quaternary ammonium) type.

These are known products. They are described for example in French Patents No. 2 505 348 or 2 542 997. Mention may be made, among the said polymers, of:

(1) Homopolymers or copolymers derived from acrylic or methacrylic esters or amides comprising at least one of the units of following formulae (II), (III), (IV) and (V):

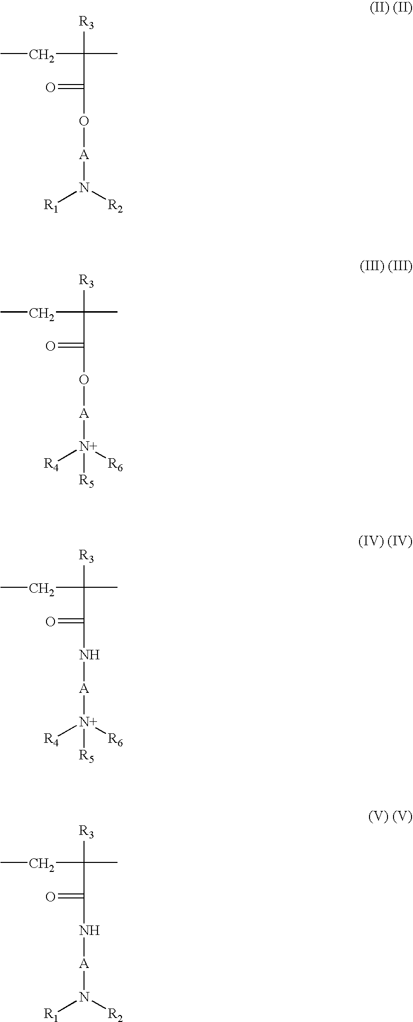

in which:

R₃, which are identical or different, denote a hydrogen atom or a CH₃ radical;

A, which are identical or different, represent a linear or branched alkyl group comprising from 1 to 6 carbon atoms, such as 2 or 3 carbon atoms, or a hydroxyalkyl group comprising from 1 to 4 carbon atoms;

R₄, R₅ and R₆, which are identical or different, represent an alkyl group having from 1 to 18 carbon atoms or a benzyl radical and such as an alkyl group having from 1 to 6 carbon atoms;

R₁ and R₂, which are identical or different, represent hydrogen or an alkyl group having from 1 to 6 carbon atoms and such as methyl or ethyl;

X denotes an anion derived from an inorganic or organic acid, such as a methyl sulphate anion or a halide, such as chloride or bromide.

The polymers of family (1) can additionally comprise at least one unit deriving from comonomers which can be chosen from the family of the acrylamides, methacrylamides, diacetone acrylamides, acrylamides, and methacrylamides substituted on the nitrogen by lower (C1-C4) alkyls, acrylic or methacrylic acids or their esters, vinyllactams, such as vinylpyrrolidone or vinylcaprolactam, or vinyl esters.

Thus, mention may be made, among these polymers of family (1), of:

copolymers of acrylamide and of dimethylaminoethyl methacrylate which is quaternized with dimethyl sulphate or with a methyl halide;

copolymers of acrylamide and of methacryloyloxyethyltrimethylammonium chloride, described, for example, in Patent Application EP-A-080 976;

copolymer of acrylamide and of methacryloyloxyethyltrimethylammonium methyl sulphate;

vinylpyrrolidone/dialkylaminoalkyl acrylate or methacrylate copolymers, which may or may not be quaternized. These polymers are described in detail in French Patents 2 077 143 and 2 393 573;

dimethylaminoethyl methacrylate/vinylcaprolactam/vinylpyrrolidone terpolymers;

vinylpyrrolidone/methacrylamidopropyldimethylamine copolymers;

vinylpyrrolidone/quaternized dimethylaminopropylmethacrylamide copolymers;

crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride or by copolymerization of acrylamide with dimethylaminoethyl methacrylate quaternized with methyl chloride, the homo- or copolymerization being followed by a crosslinking with a compound comprising olefinic unsaturation, for example methylene-bisacrylamide. Use may for example be made of an acrylamide/methacryloyloxyethyltrimethylammonium chloride (20/80 by weight) crosslinked copolymer in the form of a dispersion comprising 50% by weight of the said copolymer in mineral oil. This dispersion is sold under the name of “SALCARE® SC 92” by Ciba. Use may also be made of a crosslinked homopolymer of methacryloyloxyethyltrimethyl-ammonium chloride comprising approximately 50% by weight of the homopolymer in mineral oil or in a liquid ester. These dispersions are sold under the names of “SALCARE®SC 95” and “SALCARE® SC 96” by Ciba.

(2) Cationic polysaccharides and for example those chosen from:

(a) the cellulose ether derivatives comprising quaternary ammonium groups described in French Patent 1 492 597. These polymers are also defined in the CTFA dictionary as quaternary ammoniums of hydroxyethylcellulose having reacted with an epoxide substituted by a trimethylammonium group.

(b) the cellulose copolymers or the cellulose derivatives grafted with a water-soluble quaternary ammonium monomer and described for example in U.S. Pat. No. 4,131,576, such as hydroxyalkylcelluloses, for example hydroxymethyl-, hydroxyethyl- or hydroxypropylcelluloses, grafted for example with a methacryloyloxyethyltrimethylammonium, methacrylamidopropyltrimethylammonium or dimethyldiallylammonium salt.

(c) the cationic polygalactomannans described for example in U.S. Pat. Nos. 3,589,578 and 4,031,307, such as guar gums comprising trialkylammonium cationic groups. Use is made, for example, of guar gums modified by a 2,3-epoxypropyl-trimethylammonium salt (e.g. chloride).

(3) Polymers composed of piperazinyl units and of divalent straight- or branched-chain alkylene or hydroxyalkylene radicals, optionally interrupted by oxygen, sulphur or nitrogen atoms or by aromatic or heterocyclic rings, as well as the oxidation and/or quaternization products of these polymers. Such polymers are described for example in French Patents 2 162 025 and 2 280 361.

(4) Water-soluble polyaminoamides prepared for example by polycondensation of an acidic compound with a polyamine; these polyaminoamides can be crosslinked by an epihalohydrin, a diepoxide, a dianhydride, an unsaturated dianhydride, a bisunsaturated derivative, a bishalohydrin, a bisazetidinium, a bishaloacyldiamine or an alkyl bishalide or alternatively by an oligomer resulting from the reaction of a bifunctional compound reactive with respect to a bishalohydrin, a bisazetidinium, a bishaloacyldiamine, an alkyl bishalide, an epihalohydrin, a diepoxide or a bisunsaturated derivative; the crosslinking agent being used in an amount ranging from 0.025 to 0.35 mol per amine group of the polyaminoamide; these polyaminoamides can be alkylated or, if they comprise at least one tertiary amine functional group, quaternized. Such polymers are described for example in French Patents 2 252 840 and 2 368 508.

(5) Polyaminoamide derivatives resulting from the condensation of polyalkylenepolyamines with polycarboxylic acids, followed by an alkylation by bifunctional agents. Mention may be made, for example, of adipic acid/dialkylaminohydroxyalkyldialkylenetriamine polymers in which the alkyl radical comprises from 1 to 4 carbon atoms and for example denotes methyl, ethyl, or propyl. Such polymers are described for example in French Patent 1 583 363.

Mention may for example be made, among these derivatives, of adipic acid/dimethylaminohydroxypropyl/diethylenetriamine polymers.

(6) Polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms; the molar ratio of polyalkylenepolyamine to dicarboxylic acid ranging from 0.8:1 to 1.4:1; the polyaminoamide resulting therefrom being reacted with epichlorohydrin in a molar ratio of epichlorohydrin with respect to the secondary amine group of the polyaminoamide ranging from 0.5:1 to 1.8:1. Such polymers are described for example in U.S. Pat. Nos. 3,227,615 and 2,961,347.

(7) Cyclopolymers of alkyldiallylamine or of dialkyldiallylammonium, such as the homopolymers or copolymers comprising, as main constituent of the chain, units corresponding to the formulae (VI) or (VII):

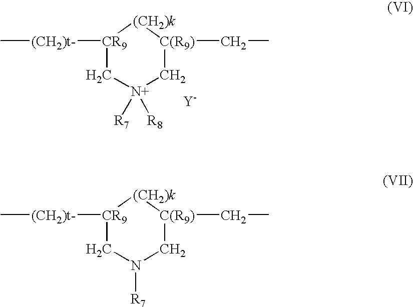

in which formulae k and t are equal to 0 or 1, the sum k+t being equal to 1; R₉ denotes a hydrogen atom or a methyl radical; R₇ and R₈, independently of one another, denote an alkyl group having from 1 to 6 carbon atoms, a hydroxyalkyl group in which the alkyl group for example has 1 to 5 carbon atoms or a lower (C₁-C₄)amidoalkyl group or R₇ and R₈ can denote, jointly with the nitrogen atom to which they are attached, heterocyclic groups, such as piperidinyl or morpholinyl; R₇ and R₈, independently of one another, for example denote an alkyl group having from 1 to 4 carbon atoms; Y⁻ is an anion, such as bromide, chloride, acetate, borate, citrate, tartrate, bisulphate, bisulphite, sulphate or phosphate. These polymers are described for example in French Patent 2 080 759 and in its certificate of addition 2 190 406.

(8) The quaternary diammonium polymer comprising repeat units corresponding to the formula:

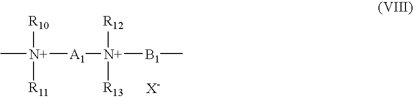

in which formula (VIII):

R₁₀, R₁₁, R₁₂ and R₁₃, which are identical or different, represent aliphatic, alicyclic or arylaliphatic radicals comprising from 1 to 20 carbon atoms or lower aliphatic hydroxyalkyl radicals or else R₁₀, R₁₁, R₁₂ and R₁₃, together or separately, form, with the nitrogen atoms to which they are attached, heterocycles optionally comprising a second heteroatom other than nitrogen or else R₁₀, R₁₁, R₁₂ and R₁₃ represent a linear or branched C₁-C₆ alkyl radical substituted by a nitrile, ester, acyl, amide or —CO—O—R₁₄-D or —CO—NH—R₁₄-D group, where R₁₄ is an alkylene and D a quaternary ammonium group;

A₁ and B₁ represent polymethylene groups comprising from 2 to 20 carbon atoms which can be linear or branched and saturated or unsaturated, and which can comprise, bonded to or inserted into the main chain, at least one aromatic ring or at least one oxygen or sulphur atom or sulphoxide, sulphone, disulphide, amino, alkylamino, hydroxyl, quaternary ammonium, ureido, amide or ester groups, and

X⁻ denotes an anion derived from an inorganic or organic acid;

A₁, R₁₀, and R₁₂ can form, with the two nitrogen atoms to which they are attached, a piperazine ring; in addition, if A₁ denotes a linear or branched and saturated or unsaturated alkylene or hydroxyalkylene radical, B₁ can also denote a —(CH₂)ₙ—CO-D-OC—(CH₂)ₙ— group in which n ranges from 1 to 100 and such as from 1 to 50 and D denotes:

a) a glycol residue of formula: —O—Z—O—, where Z denotes a linear or branched hydrocarbon radical or a group corresponding to one of the following formulae:

—(CH₂—CH₂—O)ₓ—CH₂—CH₂

—[CH₂—CH(CH₃)—O]_{y}—CH₂—CH(CH₃)—

where x and y denote an integer from 1 to 4, representing a defined and unique degree of polymerization, or any number from 1 to 4 representing a mean degree of polymerization;

b) a bissecondary diamine residue, such as a piperazine derivative;

c) a bisprimary diamine residue of formula: —NH—Y—NH—, where Y denotes a linear or branched hydrocarbon radical or else the divalent radical

—CH₂—CH₂—S—S—CH₂—CH₂—;

d) a ureylene group of formula: —NH—CO—NH—.

For example, X⁻ may be an anion, such as chloride or bromide.

These polymers have a number-average molecular weight generally ranging from 1,000 to 100,000.

Polymers of this type are described for example in French Patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may for example be made of the polymers which are composed of repeat units corresponding to the following formula (IX):

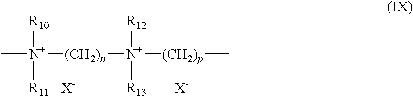

in which R₁₀, R₁₁, R₁₂, and R₁₃, which are identical or different, denote an alkyl or hydroxyalkyl radical having from 1 to 4 carbon, n and p may be integers varying from 2 to 20 and X⁻ may be an anion derived from an inorganic or organic acid.

(9) Polymers of poly(quaternary ammonium)s composed of repeat units of formula (X):

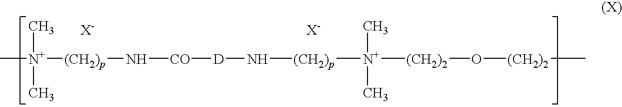

in which p denotes an integer varying from 1 to 6, D may not exist or may represent a —(CH₂)ᵣ—CO— group in which r denotes a number equal to 4 or to 7, and X⁻ is an anion.

Such polymers can be prepared according to the processes described in U.S. Pat. Nos. 4,157,388, 4,702,906 and 4,719,282. They are described for example in Patent Application EP-A-122 324.

(10) Quaternary polymers of vinylpyrrolidone and of vinylimidazole.

(11) Polyamines, such as the product referenced under the name of “Polyethylene Glycol (15) Tallow Polyamine” in the CTFA dictionary.

Other cationic polymers which can be used in the context of the disclosure are polyalkyleneimines, such as polyethyleneimines, polymers comprising vinylpyridine or vinylpyridinium units, condensates of polyamines and of epichlorohydrin, quaternary polyureylenes and chitin derivatives.

For example, among the cationic polymers which can be used according to the present disclosure, mention may be made of:

quaternary cellulose ether derivatives, such as the products sold under the name “JR 400” by Rhodia Chimie, cyclopolymers, for example diallyldimethylammonium salt homopolymers and diallyldimethylammonium salt and acrylamide copolymers, for example the chlorides, sold under the names “MERQUAT 550” and “MERQUAT S” by Merck, cationic polysaccharides and such as guar gums modified by 2,3-epoxypropyltrimethylammonium chloride, for example sold under the name “JAGUAR C13S” by Rhodia Chimie, optionally crosslinked homopolymers and copolymers of (meth)acryloyloxyethyltrimethylammonium salt, sold by Ciba in solution at 50% in mineral oil under the trade names SALCARE SC92 (crosslinked copolymer of methacryloyloxyethyltrimethylammonium chloride and of acrylamide) and SALCARE SC95 (crosslinked homopolymer of methacryloyloxyethyltrimethylammonium chloride), or quaternary copolymers of vinylpyrrolidone and of vinylimidazole salt, such as the products sold by BASF under the names LUVIQUAT FC 370, LUVIQUAT FC 550, LUVIQUAT FC 905 and LUVIQUAT HM-552.

According to at least one embodiment, the compositions according to the disclosure comprise at least one cationic polymer chosen from dimethyldiallylammonium chloride homopolymers and copolymers.

The amphoteric polymers which can be used in the compositions of the present disclosure can be chosen from polymers comprising K and M units distributed randomly in the polymer chain, where K denotes a unit deriving from a monomer comprising at least one basic nitrogen atom and M denotes a unit deriving from an acidic monomer comprising at least one carboxyl or sulpho group or else K and M can denote groups deriving from zwitterionic carboxybetaine or sulphobetaine monomers.

K and M can also denote a cationic polymer chain comprising primary, secondary, tertiary or quaternary amine groups, in which chain at least one of the amine groups carries a carboxyl or sulpho group connected via a hydrocarbon radical, or else K and M form part of a chain of a polymer comprising an α,β-dicarboxyethylene unit, one of the carboxyl groups of which has been reacted with a polyamine comprising at least one primary or secondary amine group.

For example, the amphoteric polymer corresponding to the definition given above may be chosen from the following polymers:

(1) Polymers resulting from the copolymerization of a monomer derived from a vinyl compound carrying a carboxyl group, such as acrylic acid, methacrylic acid, maleic acid or α-chloroacrylic acid, and of a basic monomer derived from a substituted vinyl compound comprising at least one basic atom, such as dialkylaminoalkyl methacrylates and acrylates or dialkylaminoalkylmethacrylamides and -acrylamides. Such compounds are described in U.S. Pat. No. 3,836,537. Mention may also be made of sodium acrylate/acrylamidopropyltrimethylammonium chloride copolymer.

The vinyl compound can also be a dialkyldiallylammonium salt, such as dimethyldiallylammonium chloride.

(2) Polymers comprising units deriving:

a) from at least one monomer chosen from acrylamides or methacrylamides substituted on the nitrogen by an alkyl radical,

b) from at least one acidic comonomer comprising at least one reactive carboxyl groups, and

c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulphate.

For example, the N-substituted acrylamides or methacrylamides according to the disclosure can be the compounds in which the alkyl radicals comprise from 2 to 12 carbon atoms and for example N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers may for example be chosen from acrylic, methacrylic, crotonic, itaconic, maleic or fumaric acids, and alkyl monoesters having 1 to 4 carbon atoms of maleic or fumaric acids or anhydrides.

For example, the basic comonomers can be aminoethyl, butylaminoethyl, N,N-dimethylaminoethyl, or N-tert-butylaminoethyl methacrylates.

(3) Partially or completely alkylated and crosslinked polyaminoamides deriving from polyaminoamides of general formula:

CO—R₁₉—CO—Z (XI)

in which R₁₉ represents a divalent radical derived from a saturated dicarboxylic acid, from an aliphatic mono- or dicarboxylic acid comprising an ethylenic double bond, from an ester of a lower alkanol having 1 to 6 carbon atoms of these acids, or from a radical deriving from the addition of any one of the said acids with a bisprimary or bissecondary amine, and Z denotes a radical of a bisprimary, mono- or bissecondary polyalkylenepolyamine and for example represents:

a) in an amount ranging from 60 to 100 mol %, the radical

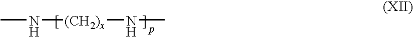

where x=2 and p=2 or 3, or else x=3 and p=2

this radical deriving from diethylenetriamine, triethylenetetraamine or dipropylenetriamine;

b) in an amount ranging from 0 to 40 mol %, the above radical (XII), in which x=2 and p=1 and which derives from ethylenediamine, or the radical deriving from piperazine:

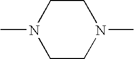

c) in an amount ranging from 0 to 20 mol %, the radical —NH—(CH₂)₆—NH— deriving from hexamethylenediamine, these polyaminoamides being crosslinked by addition of a bifunctional crosslinking agent chosen from epihalohydrins, diepoxides, dianhydrides, or bisunsaturated derivatives, via from 0.025 to 0.35 mol of crosslinking agent per amine group of the polyaminoamide, and alkylated by reaction with acrylic acid, chloroacetic acid or an alkanesultone or their salts.

The saturated carboxylic acids may be for example chosen from acids having 6 to 10 carbon atoms, such as adipic, 2,2,4-trimethyladipic, and 2,4,4-trimethyladipic, or terephthalic acid, and the acids comprising an ethylenic double bond, such as, for example, acrylic, methacrylic or itaconic acids.

The alkanesultones used in the alkylation may be for example propane- or butanesultone and the salts of the alkylating agents may be for example the sodium or potassium salts.

(4) Polymers comprising zwitterionic units of formula:

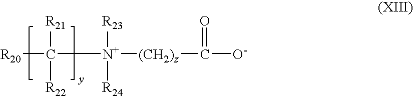

in which R₂₀ denotes a polymerizable unsaturated group, such as an acrylate, methacrylate, acrylamide, or methacrylamide group, y and z represent an integer from 1 to 3, R₂₁ and R₂₂ represent a hydrogen atom or methyl, ethyl, or propyl, and R₂₃ and R₂₄ represent a hydrogen atom or an alkyl radical such that the sum of the carbon atoms in R₂₃ and R₂₄ does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Mention may be made, by way of example, of butyl methacrylate/dimethylcarboxymethylammonioethyl methacrylate copolymer, such as the product sold under the name DIAFORMER Z301 by Sandoz.

(5) Polymers derived from chitosan comprising monomer units corresponding to the following formulae (XIII), (XIV) and (XV):

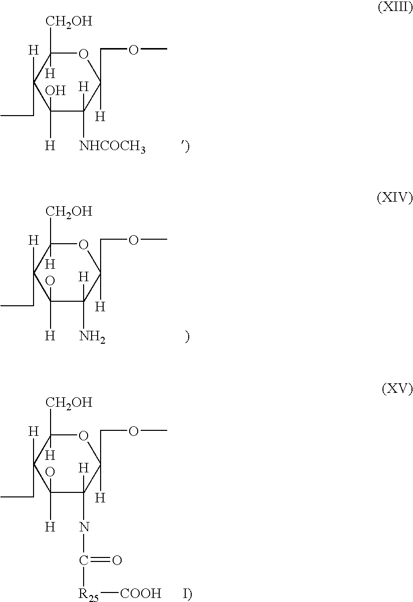

the unit (XIII) being present in an amount ranging from 0 to 30%, the unit (XIV) in an amount ranging from 5 to 50% and the unit (XV) in an amount ranging from 30 and 90%, it being understood that, in this unit (XV), R₂₅ represents a radical of formula:

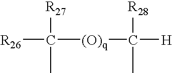

in which q denotes zero or 1; if q=0, R₂₆, R₂₇, and R₂₈, which are identical or different, each represent a hydrogen atom, a methyl, hydroxyl, acetoxy, or amino residue, a monoalkylamino residue or a dialkylamino residue, optionally interrupted by at least one nitrogen atom and/or optionally substituted by at least one amino, hydroxyl, carboxyl, alkylthio or sulpho group, or an alkylthio residue in which the alkyl group carries an amino residue, at least one of the R₂₆, R₂₇ and R₂₈ radicals being, in this case, a hydrogen atom;

or, if q=1, R₂₆, R₂₇ and R₂₈ each represent a hydrogen atom, and the salts formed by these compounds with bases or acids.

(6) Polymers derived from the N-carboxyalkylation of chitosan, such as the N-(carboxymethyl)chitosan or N-(carboxybutyl)chitosan.

(7) Polymers corresponding to the general formula (XVI), such as those described, for example, in French Patent 1 400 366 and comprising units:

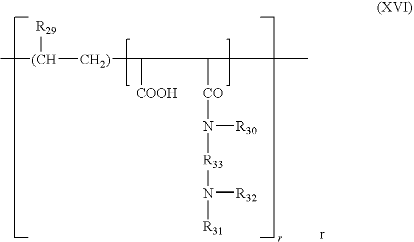

in which R₂₉ represents a hydrogen atom or a CH₃O, CH₃CH₂O or phenyl radical, R₃₀ denotes hydrogen or a lower alkyl radical, such as methyl or ethyl, R₃₁ denotes hydrogen or a lower alkyl radical, such as methyl or ethyl, and R₃₂ denotes a lower alkyl radical, such as methyl or ethyl, or a radical corresponding to the formula: —R₃₃—N(R₃₁)₂, R₃₃ representing a —CH₂—CH₂—, —CH₂—CH₂—CH₂— or —CH₂—CH(CH₃)— group and R₃₁ having the meanings mentioned above,

as well as the higher homologues of these radicals comprising up to 6 carbon atoms;

r is such that the molecular weight ranges from 500 to 6,000,000 such as from 1,000 to 1,000,000.

(8) Amphoteric polymers of the -D-X-D-X— type chosen from:

a) polymers obtained by reaction of chloroacetic acid or sodium chloroacetate with compounds comprising at least one unit of formula:

-D-X-D-X-D- (XVII)

where D denotes a radical

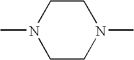

and X denotes the symbol E or E′, E or E′, which are identical or different, denoting a bivalent radical which is a straight- or branched-chain alkylene radical comprising up to 7 carbon atoms in the main chain which is unsubstituted or substituted by hydroxyl groups and which can additionally comprise oxygen, nitrogen or sulphur atoms or 1 to 3 aromatic and/or heterocyclic rings; the oxygen, nitrogen, and sulphur atoms being present in the form of ether, thioether, sulphoxide, sulphone, sulphonium, alkylamine, or alkenylamine groups or hydroxyl, benzylamine, amine oxide, quaternary ammonium, amide, imide, alcohol, ester, and/or urethane groups;

b) polymers of formula (XVIII):

-D-X-D-X— (XVIII)

where D denotes a radical

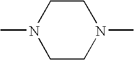

and X denotes the symbol E or E′ and E′ at least once, E having the meaning indicated above and E′ being a bivalent radical which is a straight- or branched-chain alkylene radical having up to 7 carbon atoms in the main chain which is substituted or unsubstituted by at least one hydroxyl radical and which comprises at least one nitrogen atom, the nitrogen atom being substituted by an alkyl chain optionally interrupted by an oxygen atom and necessarily comprising at least one carboxyl functional group or at least one hydroxyl functional group and betainized by reaction with chloroacetic acid or sodium chloroacetate.

(9) (C₁-C₅)Alkyl vinyl ether/maleic anhydride copolymers partially modified by semiamidation with an N,N-dialkylaminoalkylamine, such as N,N-dimethylaminopropylamine, or by semiesterification with an N,N-dialkanolamine. These copolymers can also comprise other vinyl comonomers, such as vinylcaprolactam.

For example, among all the amphoteric polymers which can be used in the composition according to the present disclosure, to:

the dimethyldiallylammonium chloride/acrylic acid (80/20) copolymer sold under the name MERQUAT 280 Dry by Calgon (CTFA name: POLYQUATERNIUM 22);

the dimethyldiallylammonium chloride/acrylic acid (95/5) copolymer sold under the name MERQUAT 295 Dry by Calgon (CTFA name: POLYQUATERNIUM 22);

the copolymer of methacrylamidopropyltrimonium chloride, of acrylic acid and of ethyl acrylate sold under the name MERQUAT 2001 by Calgon (CTFA name: POLYQUATERNIUM 47); and

the acrylamide/dimethyldiallylammonium chloride/acrylic acid terpolymer sold under the name MERQUAT PLUS 3330 Dry by Calgon (CTFA name: POLYQUATERIUM 39).

The nonionic polymers which can be used according to the present disclosure are chosen, for example, from:

vinylpyrrolidone homopolymers;

vinylpyrrolidone and vinyl acetate copolymers;

polyalkyloxazolines, such as the polyethyloxazolines sold by Dow Chemical under the names PEOX 50,000; PEOX 200,000; and PEOX 500,000;

vinyl acetate homopolymers, such as the product sold under the name Appretan EM by Hoechst or the product sold under the name RHODOPAS A 012 by Rhodia Chimie;

copolymers of vinyl acetate and of acrylic ester, such as the product sold under the name RHODOPAS AD 310 by Rhodia Chimie;

copolymers of vinyl acetate and of ethylene, such as the product sold under the name APPRETAN TV by Hoechst;

copolymers of vinyl acetate and of maleic ester, for example dibutyl maleate, such as the product sold under the name APPRETAN MB Extra by Hoechst;

copolymers of polyethylene and of maleic anhydride; alkyl acrylate homopolymers and alkyl methacrylate homopolymers, such as the product sold under the name MICROPEARL RQ 750 by Matsumoto or the product sold under the name LUHYDRAN A 848 S by BASF;

acrylic ester copolymers, such as, for example, alkyl(meth)acrylate copolymers, such as the products sold by Rohm & Haas under the names PRIMAL AC-261 K and EUDRAGIT NE 30 D, by BASF under the names ACRONAL 601, LUHYDRAN LR 8833 or LUHYDRAN LR 8845 and by Hoechst under the names APPRETAN N 9213 or N 9212;

copolymers of acrylonitrile and of a nonionic monomer chosen, for example, from butadiene and alkyl(meth)acrylates; mention may be made of the products sold under the names NIPOL LX 531 B by NIPPON Zeon or those sold under the name CJ0601 B by Rohm & Haas;

polyurethanes, such as the products sold under the names ACRYSOL RM 1020 or ACRYSOL RM 2020 by Rohm & Haas and the products URAFLEX XP 401 UZ and URAFLEX XP 402 UZ sold by DSM Resins;

copolymers of alkyl acrylate and of urethane, such as the product 8538-33 sold by National Starch;

polyamides, such as the product ESTAPOR LO 11 sold by Rhodia Chimie;

chemically modified or unmodified nonionic guar gums.

The unmodified nonionic guar gums are, for example, the products sold under the name VIDOGUM GH 175 by Unipectine or under the name JAGUAR C by Meyhall.

The modified nonionic guar gums which can be used according to the disclosure are, for example, modified by C₁-C₆ hydroxyalkyl groups. Mention may be made, as examples, of the hydroxymethyl, hydroxyethyl, hydroxypropyl, and hydroxybutyl groups.

These guar gums are known in the art and can be prepared, for example, by reacting corresponding alkene oxides, such as, for example, propylene oxides, with the guar gum, so as to obtain a guar gum modified by hydroxypropyl groups.

Such nonionic guar gums optionally modified by hydroxyalkyl groups are, for example, sold under the trade names JAGUAR HP8, JAGUAR HP60, JAGUAR HP120, JAGUAR DC293 and JAGUAR HP105 by Meyhall and under the name GALACTOSOL 4H4FD2 by Aqualon.

The alkyl groups of the nonionic polymers comprise from 1 to 6 carbon atoms, unless otherwise mentioned.

It is also possible to use, as polymers, functionalized or nonfunctionalized and silicone-comprising or non-silicone-comprising polyurethanes.

Examples of polyurethanes that may be used include those disclosed in Patents EP 0 751 162, EP 0 637 600, FR 2 743 297, EP 0 648 485, EP 0 656 021, WO 94/03510, and EP 0 619 111.

In another embodiment, the polymers can be used in the dissolved form or can be in the form of dispersions of solid or liquid particles (latexes or pseudolatexes).

According to another embodiment, the at least one cosmetic adjuvant may be chosen from silicones.

In the present disclosure, silicone may be intended to denote, in conformity with what is generally accepted, any organosilicon polymer or oligomer with a linear or cyclic and branched or crosslinked structure of variable molecular weight obtained by polymerization and/or by polycondensation of suitably functionalized silanes and essentially composed of a repetition of main units in which the silicon atoms are connected to one another via oxygen atoms (siloxane —Si—O—Si— bond), optionally substituted hydrocarbon radicals being directly connected via a carbon atom to the silicon atoms. The commonest hydrocarbon radicals are alkyl radicals, such as C₁-C₁₀ alkyl radicals and for example methyl radicals, fluoroalkyl radicals, the alkyl part of which is a C₁-C₁₀ alkyl radical, or aryl radicals and for example the phenyl radical.

The silicones used in the composition according to the disclosure may be volatile or nonvolatile and soluble or insoluble in the composition. They can for example be polyorganosiloxanes which are insoluble in the composition of the disclosure and which are provided in the form of oils, waxes, resins, or gums.

The insoluble silicones may be for example dispersed in the compositions in the form of particles generally having a number-average size ranging from 2 nanometres to 100 micrometres, such as ranging from 20 nanometres to 20 micrometres (measured with a particle sizer).

Organopolysiloxanes are defined in more detail in the work by Walter Noll, “Chemistry and Technology of Silicones”, (1968), Academic Press. They can be volatile or nonvolatile.

When they are volatile, the silicones may be chosen for example from those having a boiling point ranging from 60° C. to 260° C. and for example from:

(i) cyclic silicones comprising from 3 to 7 silicon atoms such as 4 or 5. They are, for example, octamethylcyclotetrasiloxane, sold in for example under the name of “VOLATILE SILICONE 7207” by Union Carbide or “SILBIONE 70045 V 2” by Rhodia, decamethylcyclopentasiloxane, sold under the name of “VOLATILE SILICONE 7158” by Union Carbide or “SILIBIONE 70045 V 5” by Rhodia, and their mixtures.

Mention may also be made of cyclocopolymers of the dimethylsiloxane/methylalkylsiloxane type, such as “SILICONE VOLATILE FZ 3109”, sold by Union Carbide, with the chemical structure:

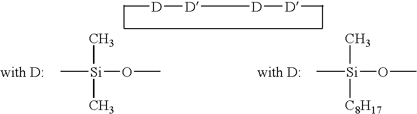

Mention may also be made of mixtures of cyclic silicones with silicon-derived organic compounds, such as the mixture of octamethylcyclotetrasiloxane and of tetratrimethylsilylpentaerythritol (50/50) and the mixture of octamethylcyclotetrasiloxane and of 1,1′-oxy(hexa-2,2,2′,2′,3,3′-trimethylsilyloxy)bisneopentane;

(ii) linear volatile silicones having from 2 to 9 silicon atoms and having a viscosity of less than or equal to 5×10⁻⁶ m²/s at 25° C. They are, for example, decamethyltetrasiloxane, sold for example under the name “SH 200” by Toray Silicone. Silicones coming within this category are also described in the article published in Cosmetics and Toiletries, Vol. 91, Jan. 76, pp. 27-32, Todd & Byers, “Volatile Silicone Fluids for Cosmetics”.

Nonvolatile silicone is understood to mean, within the meaning of the present disclosure, any silicone having a number of silicon atoms of greater than 7.

Mention may, for example, be made, among nonvolatile silicones, of polyalkylsiloxanes, polyarylsiloxanes, polyalkylarylsiloxanes, silicone gums and resins, polyorganosiloxanes modified by organofunctional groups, polysiloxane(A)-polyoxyalkylene(B) linear block copolymers of (A-B)ₙ type where n>3, grafted silicone polymers having a nonsilicone organic backbone, composed of an organic main chain formed from organic monomers not comprising silicone, to which is grafted, inside the chain and optionally at one at least of its ends, at least one polysiloxane macromonomer, grafted silicone polymers having a polysiloxane backbone grafted with nonsilicone organic monomers, comprising a polysiloxane main chain to which is grafted, inside the chain and optionally at one at least of its ends, at least one organic macromonomer not comprising silicone, and their blends.

Mention may be made, as examples of polyalkylsiloxanes, of polydimethylsiloxanes possessing trimethylsilyl end groups having a viscosity of 5×10⁻⁶ to 2.5 m²/s at 25° C. and such as of 1×10⁻⁵ to 1 m²/s. The viscosity of the silicones is, for example, measured at 25° C. according to Standard ASTM 445 Appendix C.

Mention may be made, among these polyalkylsiloxanes, without implied limitation, of the following commercial products:

SILBIONE oils of the 47 and 70,047 series or MIRASIL oils sold by Rhone-Poulenc, such as, for example, the oil 70,047 V 500,000;

oils of the MIRASIL series sold by Rhodia;

oils of the 200 series from Dow Corning, such as, DC200 with a viscosity of 60,000 cSt;

VISCASIL oils from General Electric and some oils of the SF series (SF 96, SF 18) from General Electric.

Mention may also be made of polydimethylsiloxanes possessing dimethylsilanol end groups (Dimethiconol according to the CTFA name), such as oils of the 48 series from Rhodia.

Mention may also be made, in this category of polyalkylsiloxanes, of the products sold under the names “ABIL Wax 9800” and “ABIL Wax 9801” by Goldschmidt, which are poly(C₁-C₂₀)alkylsiloxanes.

The polyalkylarylsiloxanes can for example be chosen from linear and/or branched polydimethyl(methylphenyl)siloxanes and polydimethyldiphenylsiloxanes with a viscosity of 1×10⁻⁵ to 5×10⁻² m²/s at 25° C.

Mention may be made, among these polyalkylarylsiloxanes, by way of example, of the products sold under the following names:

SILBIONE oils of the 70,641 series from Rhodia;

oils of the RHODORSIL 70,633 and 763 series from Rhodia;

the oil DOW CORNING 556 Cosmetic Grade Fluid from Dow Corning;

silicones of the PK series from Bayer, such as the product PK20;

silicones of the PN and PH series from Bayer, such as the products PN1000 and PH1000;

some oils of the SF series from General Electric, such as SF 1023, SF 1154, SF 1250 and SF 1265.

Silicone gums which can be used in accordance with the disclosure are for example polydiorganosiloxanes having high number-average molecular weights ranging from 200,000 to 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane, tridecanes, and their mixtures.

Mention may for example be made of the following products:

polydimethylsiloxane gums,

polydimethylsiloxane/methylvinylsiloxane gums,

polydimethylsiloxane/diphenylsiloxane gums,

polydimethylsiloxane/phenylmethylsiloxane gums,

polydimethylsiloxane/diphenylsiloxane/methylvinylsiloxane gums.

Mixtures of silicones can also be employed, such as:

mixtures formed from a polydimethylsiloxane gum hydroxylated at the chain end (named dimethiconol according to the nomenclature of the CTFA dictionary) and from a cyclic polydimethylsiloxane (named cyclomethicone according to the nomenclature of the CTFA dictionary), such as the product Q2 1401 sold by Dow Corning;

mixtures formed from a polydimethylsiloxane gum with a cyclic silicone, such as the product SF 1214 Silicone Fluid from General Electric; this product is a gum SF 30, corresponding to a dimethicone, having a number-average molecular weight of 500,000, dissolved in the oil SF 1202 Silicone Fluid, corresponding to decamethylcyclopentasiloxane;

mixtures of two PDMSs with different viscosities and for example a PDMS gum and a PDMS oil, such as the product SF 1236 from General Electric. The product SF 1236 is the mixture of a gum SE 30 defined above, having a viscosity of 20 m²/s, and of an oil SF 96 with a viscosity of 5×10⁻⁶ m²/s. This product for example comprises 15% of gum SE 30 and 85% of an oil SF 96.

The organopolysiloxane resins which can be used in accordance with the disclosure are crosslinked siloxane systems including the units: R₂SiO_{2/2}, R₃SiO_{1/2}, RSiO_{3/2} and SiO_{4/2}, in which R represents a hydrocarbon group possessing 1 to 16 carbon atoms or a phenyl group.

Among these products, for example are those in which R denotes a lower C₁-C₄ alkyl radical, such as methyl, or a phenyl radical.

Mention may be made, among these resins, of the product sold under the name “DOW CORNING 593” or those sold under the names “SILICONE FLUID SS 4230” and “SILICONE FLUID SS 4267” by General Electric and which are silicones with a dimethyl/trimethylsiloxane structure.

Mention may also be made of resins of the trimethylsiloxysilicate type, sold for example under the names X22-4914, X21-5034 and X21-5037 by Shin-Etsu.

The organomodified silicones which can be used in accordance with the disclosure are silicones as defined above which comprise, in their structure, at least one organofunctional group attached via a hydrocarbon group.

Mention may be made, among organomodified silicones, of the polyorganosiloxanes comprising:

polyethyleneoxy and/or polypropyleneoxy groups optionally comprising C₆-C₂₄ alkyl groups, such as the products known as dimethicone copolyol, sold by Dow Corning under the name DC 1248, or the SILWET® L 722, L 7500, L 77 and L 711 oils from Union Carbide, and (C₁₋₂)alkyl methicone copolyol, sold by Dow Corning under the name Q2 5200;

thiol groups, such as the products sold under the names “GP 72 A” and “GP 71” from Genesee;

alkoxylated groups, such as the product sold under the name “SILICONE COPOLYMER F-755” by SWS Silicones and ABIL WAX® 2428, 2434, and 2440 by Goldschmidt;

hydroxylated groups, such as the polyorganosiloxanes possessing a hydroxyalkyl functional group described in French Patent Application FR-A-85 16334;

acyloxyalkyl groups, such as, for example, the polyorganosiloxanes described in U.S. Pat. No. 4,957,732;

anionic groups of the carboxylic acid type, such as, for example, in the products described in Patent EP 186 507 from Chisso Corporation, or of the alkylcarboxyl type, such as those present in the product X-22-3701 E from Shin-Etsu; 2-hydroxyalkylsulphonate; 2-hydroxyalkylthiosulphate, such as the products sold by Goldschmidt under the names “ABIL® S201” and “ABIL® S255”;

hydroxyacylamino groups, such as the polyorganosiloxanes described in Application EP 342 834. Mention may be made, for example, of the product Q2-8413 from Dow Corning.

For example, the silicone may be an aminated silicone.

Aminated silicone is understood to mean, within the meaning of the present disclosure any silicone comprising at least one primary, secondary or tertiary amine functional group or one quaternary ammonium group.

The aminated silicones used in the composition in the film form according to the present disclosure are chosen from:

(a) the compounds corresponding to the following formula (I):

(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ (I)

in which:

T is a hydrogen atom or a phenyl, hydroxyl (—OH), C₁-C₈ alkyl, such as methyl, or C₁-C₈ alkoxy, such as methoxy, radical,

a denotes the number 0 or an integer from 1 to 3, such as 0,

b denotes 0 or 1, such as 1,

m and n are numbers such that the sum (n+m) can vary for example from 1 to 2000 and such as from 50 to 150, it being possible for n to denote a number from 0 to 1999 and such as from 49 to 149 and it being possible for m to denote a number from 1 to 2000 and such as from 1 to 10;

R¹ is a monovalent radical of formula —C_{q}H_{2q}L in which q is a number from 2 to 8 and L is an aminated group, optionally quaternized, chosen from the groups:

—N(R²)—CH₂—CH₂—N(R²)₂;

—N(R²)₂; —N⁺(R²)₃Q⁻;

—N⁺(R²)(H)₂Q⁻;

—N⁺(R²)₂HQ⁻;

—N(R²)—CH₂—CH₂—N⁺(R²)(H)₂Q⁻,

in which R² can denote a hydrogen atom, a phenyl, a benzyl, or a saturated monovalent hydrocarbon radical, for example a C₁-C₂₀ alkyl radical, and Q⁻ represents a halide ion, such as, for example, fluoride, chloride, bromide or iodide.

For example, the aminated silicones corresponding to the definition of the formula (I) are chosen from the compounds corresponding to the following formula (II):

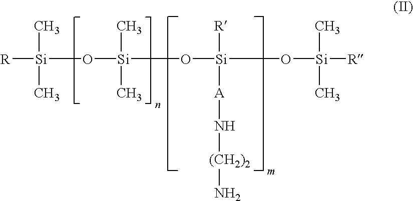

in which R, R′, and R″, which are identical or different, denote a C₁-C₄ alkyl radical, such as CH₃, a C₁-C₄ alkoxy radical, such as methoxy, or OH; A represents a linear or branched C₃-C₈, such as C₃-C₆, alkylene radical; m and n are integers depending on the molecular weight, the sum of which is between 1 and 2,000.

For example, R, R′, and R″, which are identical or different, represent a C₁-C₄ alkyl or hydroxyl radical, A represents a C₃ alkylene radical and m and n are such that the weight-average molecular weight of the compound ranges from 5,000 to 500,000. The compounds of this type are named “amodimethicone” in the CTFA dictionary.

According to another embodiment, R, R′, and R″, which are identical or different, represent a C₁-C₄ alkoxy or hydroxyl radical, one at least of the R or R″ radicals is an alkoxy radical and A represents a C₃ alkylene radical. The hydroxyl/alkoxy molar ratio may range from 0.2/1 to 0.4/1 or for example be equal to 0.3/1. Furthermore, m and n are such that the weight-average molecular weight of the compound ranges from 2,000 to 10⁶. For example, n can range from 0 to 999 and m can range from 1 to 1,000, the sum of n and m ranging from 1 and 1,000.

Mention may be made, in this category of compounds, inter alia, of the product BELSIL® ADM 652 sold by Wacker.

For example, R and R″, which are different, represent a C₁-C₄ alkoxy or hydroxyl radical, at least one of the R and R″ radicals is an alkoxy radical, R′ represents a methyl radical and A represents a C₃ alkylene radical. The hydroxyl/alkoxy molar ratio may range from 1/0.8 to 1/1.1 and for example be equal to 1/0.95. Furthermore, m and n are such that the weight-average molecular weight of the compound ranges from 2,000 to 200,000. For example, n ranges from 0 to 999 and m ranges from 1 to 1,000, the sum of n and m ranging from 1 to 1,000.

For example, mention may be made of the product FLUID WR® 1300 sold by Wacker.

It should be noted that the molecular weight of these silicones is determined by gel permeation chromatography (ambient temperature, polystyrene standard; styragem μ columns; eluent THF; flow rate of 1 mm/m; 200 μl of a 0.5% by weight solution of silicone in THF are injected and detection is carried out by refractometry and UV spectrometry).

A product corresponding to the definition of the formula (I) is for example the polymer named “trimethylsilylamodimethicone” in the CTFA dictionary, corresponding to the following formula (III):

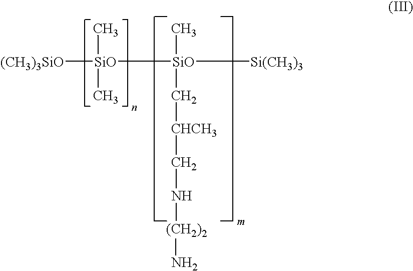

in which n and m have the meanings given above in accordance with the formula (II).

Such compounds are described, for example, in EP 95 238; a compound of formula (I) is, for example, sold under the name Q2-8220 by OSI.

(b) the compounds corresponding to the following formula (IV):

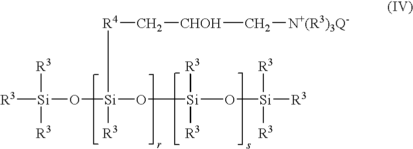

in which:

R³ represents a monovalent C₁-C₁₈ hydrocarbon radical and for example a C₁-C₁₈ alkyl or C₂-C₁₈ alkenyl radical, for example methyl;

R⁴ represents a divalent hydrocarbon radical, such as a C₁-C₁₈ alkylene radical, or a divalent C₁-C₁₈, for example C₁-C₈, alkyleneoxy radical;

Q⁻ is a halide ion, such as chloride;

r represents a mean statistical value ranging from 2 to 20 such as from 2 to 8;

s represents a mean statistical value ranging from 20 to 200 such as 20 to 50.

Such compounds are described for example in U.S. Pat. No. 4,185,087.

A compound coming within this category is that sold by Union Carbide under the name “UCAR SILICONE ALE 56”.

c) the quaternary ammonium silicones of formula (V):

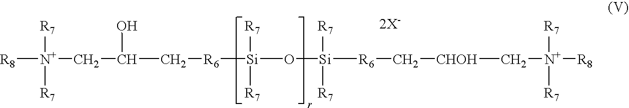

in which:

R₇, which are identical or different, represent a monovalent hydrocarbon radical having from 1 to 18 carbon atoms, such as a C₁-C₁₈ alkyl radical or a C₂-C₁₈ alkenyl radical, or a ring comprising 5 or 6 carbon atoms, for example methyl;

R₆ represents a divalent hydrocarbon radical, for example a C₁-C₁₈ alkylene radical, or a divalent C₁-C₁₈, for example C₁-C₈, alkyleneoxy radical connected to the Si via an Si—C bond;

R₈, which are identical or different, represent a hydrogen atom, a monovalent hydrocarbon radical having from 1 to 18 carbon atoms, for example a C₁-C₁₈ alkyl radical or a C₂-C₁₈ alkenyl radical, or an —R₆—NHCOR₇ radical;

X⁻ is an anion, such as a halide ion, further such as chloride, or an organic acid anion (acetate, and the like);

r represents a mean statistical value ranging from 2 to 200 such as from 5 to 100.

These silicones are, for example, described in Application EP-A-0 530 974.

According to the disclosure, use may also be made of polymers of the grafted silicone type comprising a polysiloxane portion and a portion comprising a nonsilicone organic chain, one of the two portions constituting the main chain of the polymer and the other being grafted to the main chain. These polymers are disclosed, for example, in the documents EPA 0 412 704, EP-A-0 412 707, EP-A-0 640 105, WO 95/00578, EP-A-0 582 152 and WO 93/23009 and U.S. Pat. Nos. 4,693,935, 4,728,571 and 4,972,037. These polymers may be, for example, anionic or nonionic.

Such polymers may be, for example, copolymers capable of being obtained by radical polymerization from the mixture of monomers comprising:

a) from 50% to 90% by weight of tert-butyl acrylate;

b) from 0% to 40% by weight of acrylic acid;

c) from 5% to 40% by weight of silicone macromer of formula:

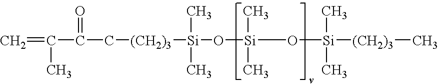

in which v is a number ranging from 5 to 700, the percentages by weight being calculated with respect to the total weight of the monomers.

Other examples of grafted silicone polymers may be polydimethylsiloxanes (PDMS) to which are grafted, via a connecting link of thiopropylene type, mixed polymer units of the poly((meth)acrylic acid) type and of the poly(alkyl(meth)acrylate) type and polydimethylsiloxanes (PDMS) to which are grafted, via a connecting link of thiopropylene type, polymer units of the poly(isobutyl(meth)acrylate) type.

For example, the at least one cosmetic adjuvant may be chosen from cationic surfactants other than the compounds of formula (I), cationic polymers and aminated silicones.

The at least one cosmetic adjuvant may be present in a total amount ranging from 0.01 to 50% by weight, such as from 0.05 to 40% by weight, further such as from 0.1 to 30% by weight, with respect to the total weight of the composition.

Cosmetically acceptable medium is understood to mean a medium compatible with keratinous substances such as the hair.

The cosmetically acceptable medium can be an alcoholic, aqueous or aqueous/alcoholic medium. Thus, the medium may be composed solely of water or of alcohol or of a mixture of water and of at least one cosmetically acceptable solvent, such as lower C₁-C₄ alcohols, polyols, polyol monoethers and their mixtures. For example, the alcohol can be ethanol.

For example, the cosmetic composition may be a rinse-out composition. “Rinse-out composition” is understood to mean any composition which is formulated in order to be rinsed out immediately or after a leave-in time of less than 30 minutes, such as less than 10 minutes, after application to the hair.

The rinse-out composition can be provided in any conventional form of rinse-out cosmetic composition, including, but without being limited thereto, shampoos, conditioners, hair rinsing lotions, perming compositions, hair dyeing compositions, products to be used before or after a hair dyeing treatment, products to be used before or after a perming treatment, hair straightening compositions, products to be used before or after a hair straightening treatment, and combinations of these. A shampoo exhibits a cleaning effect on the hair and can also exhibit a conditioning effect. A conditioner exhibits a conditioning effect on the hair without a marked cleaning effect.

For example, the composition according to the disclosure is a rinse-out composition, such as a shampoo or a conditioner.

Another aspect of the present disclosure is at least one entity chosen from from the lysine-derived compounds corresponding to the formula (I) chosen from:

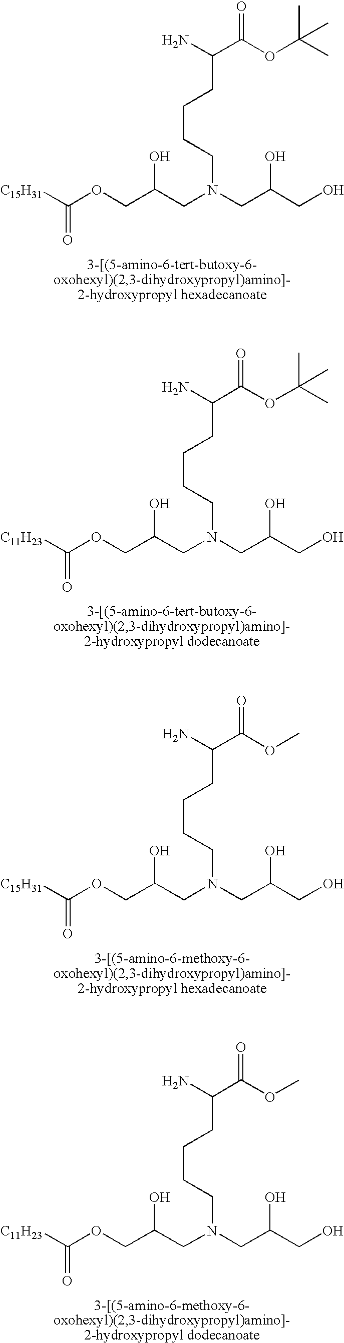

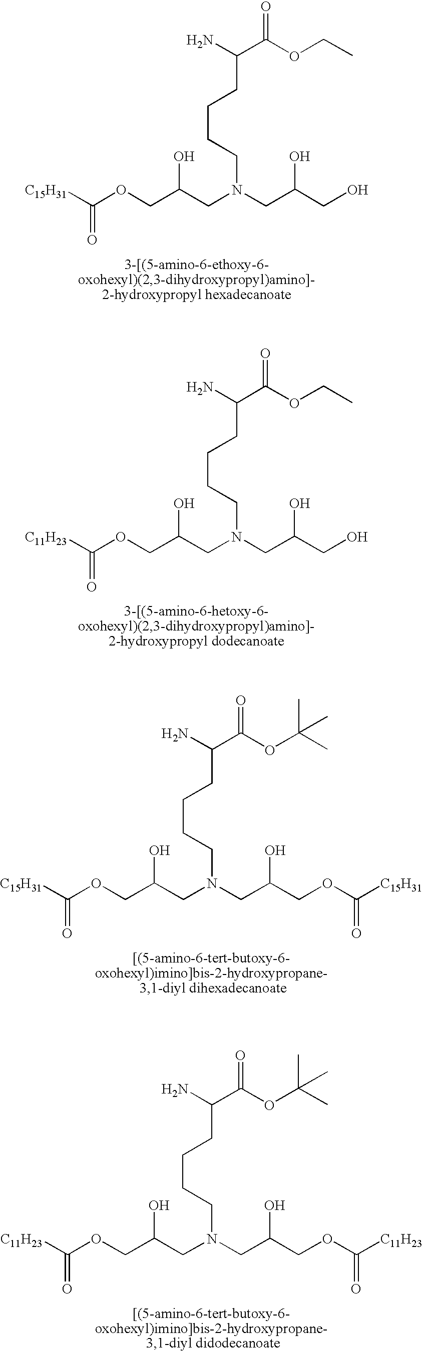

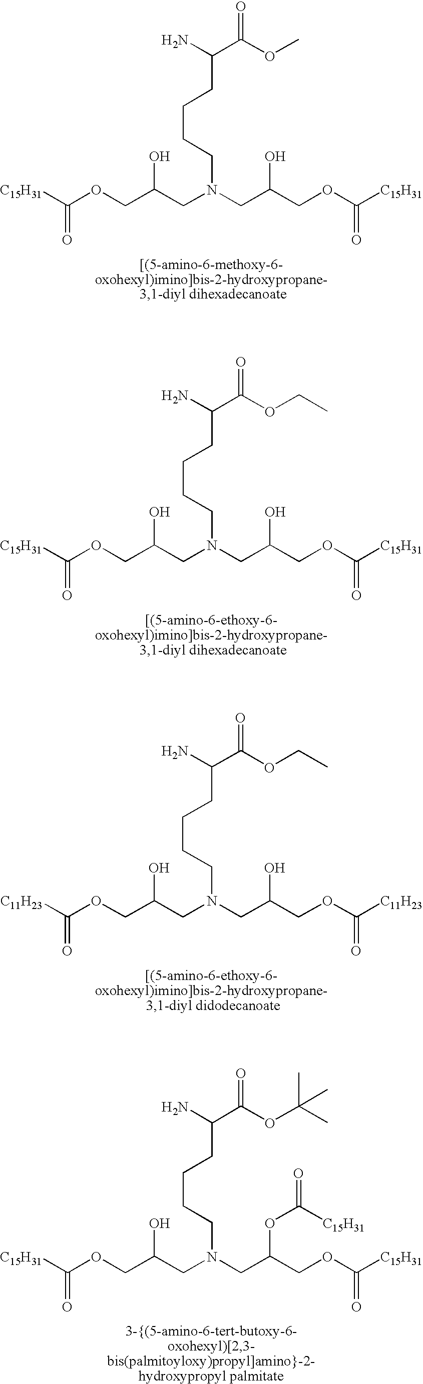

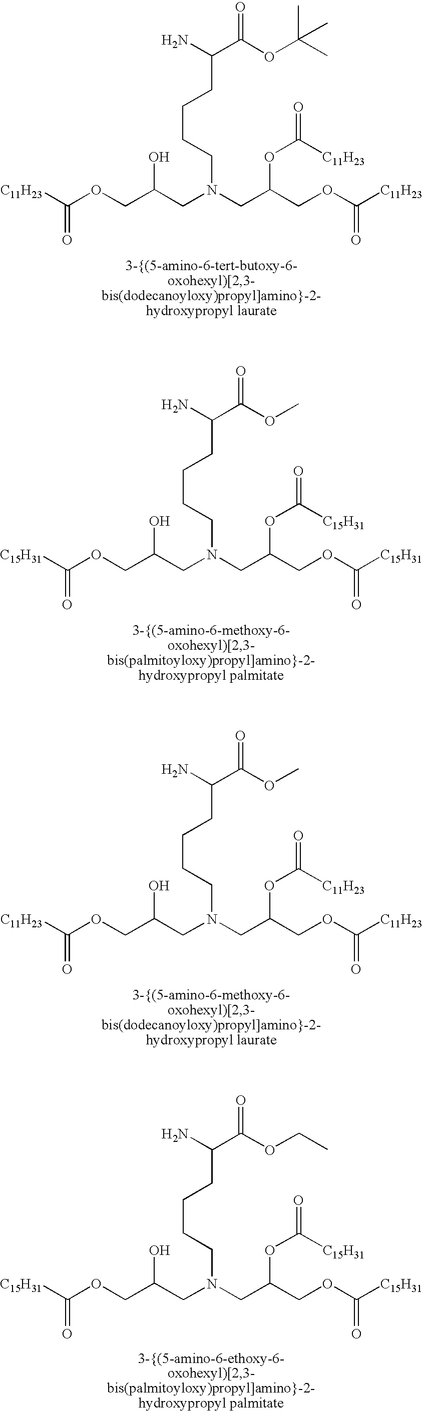

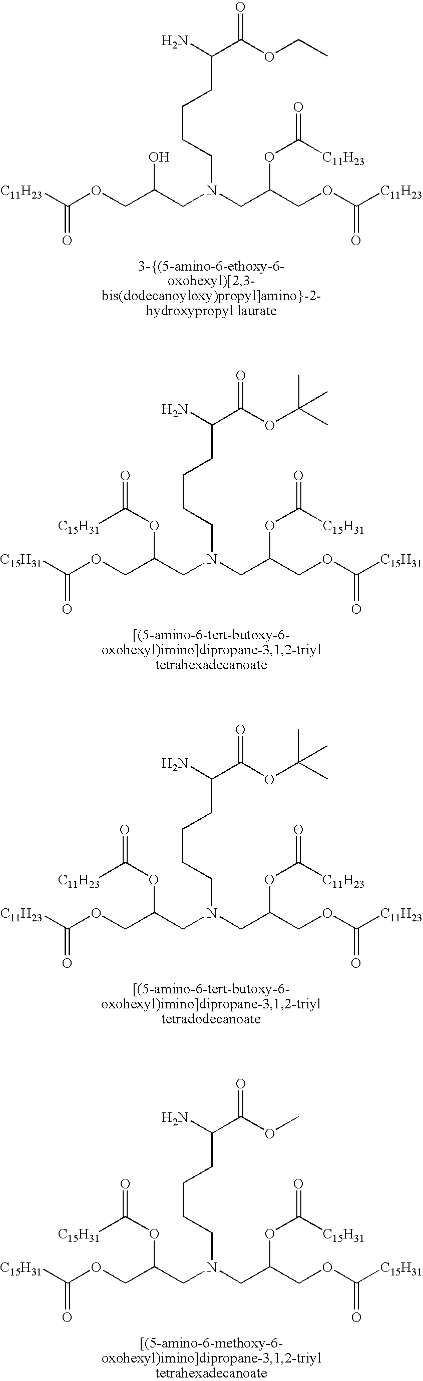

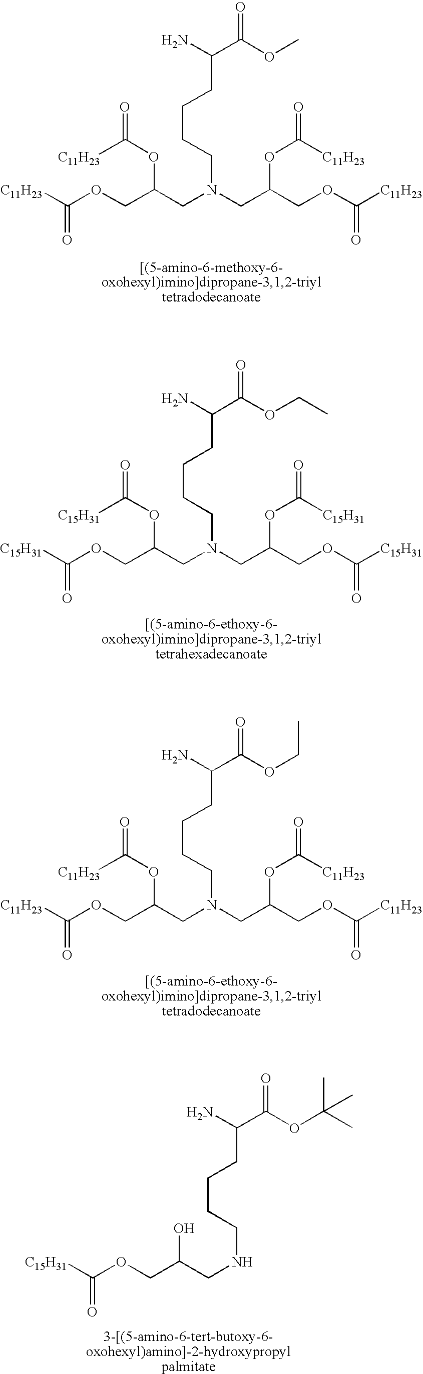

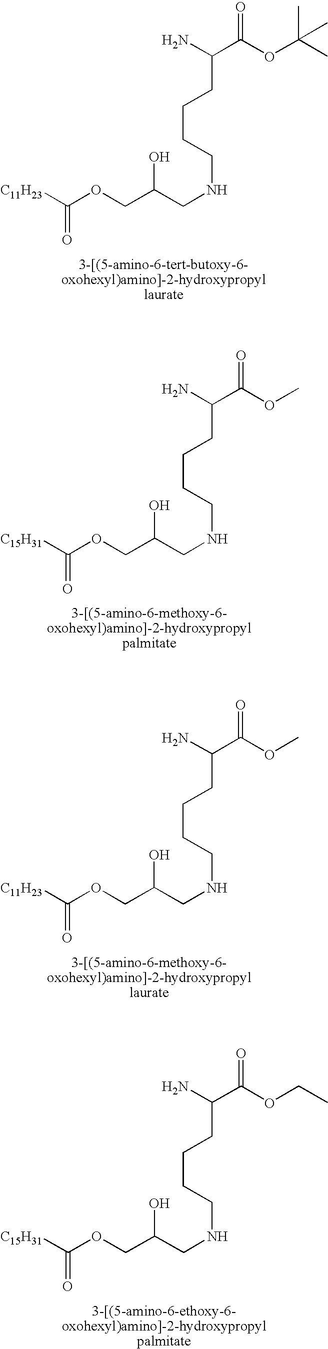

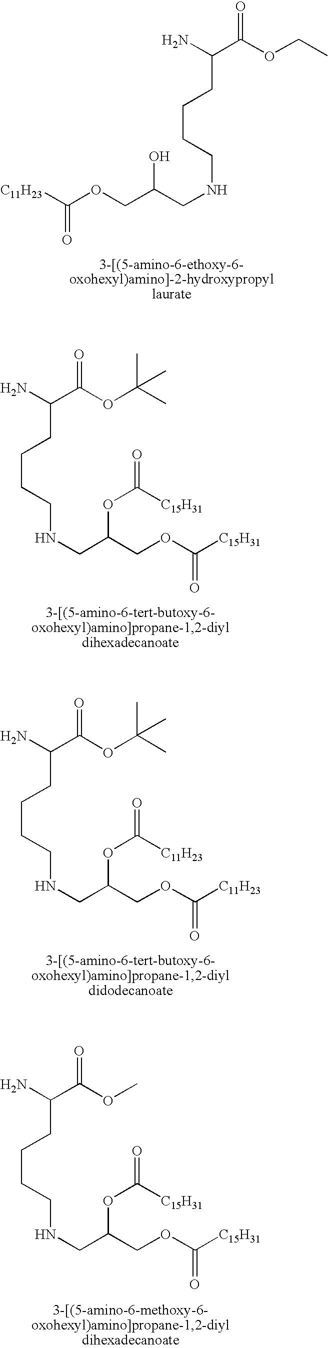

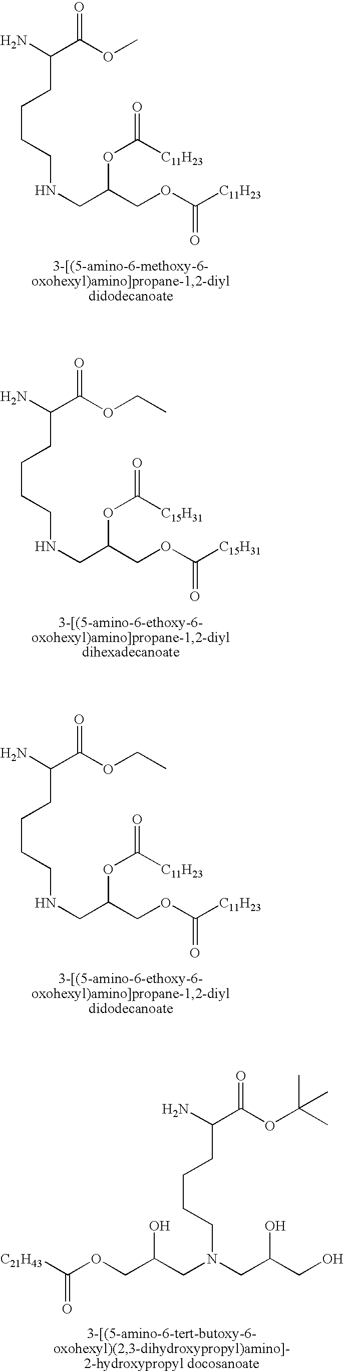

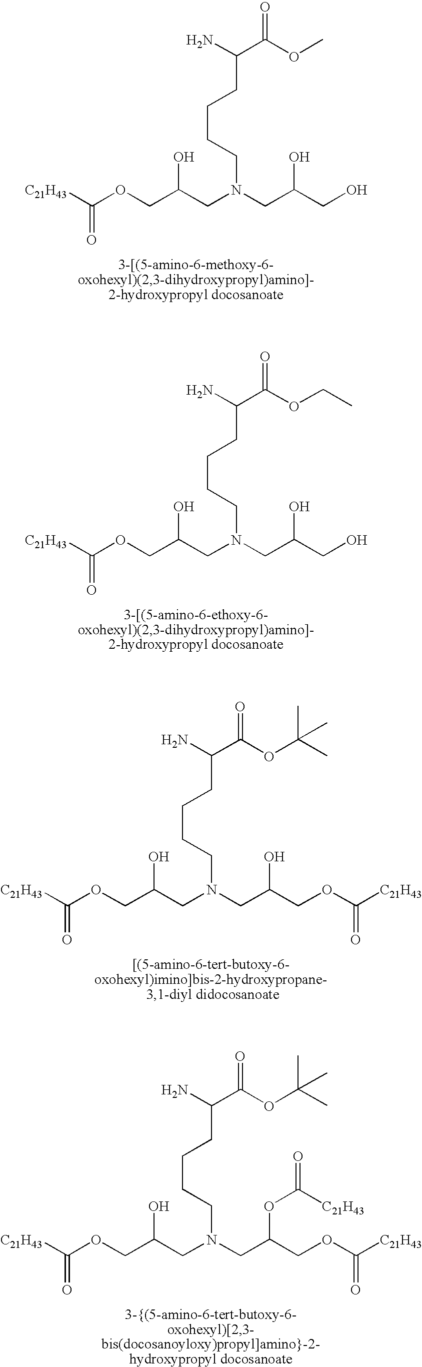

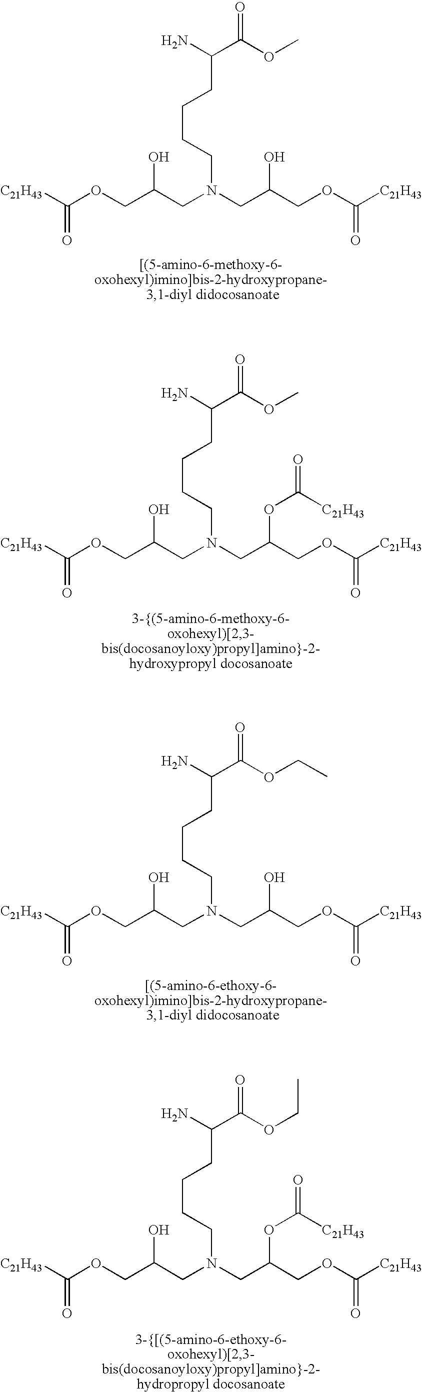

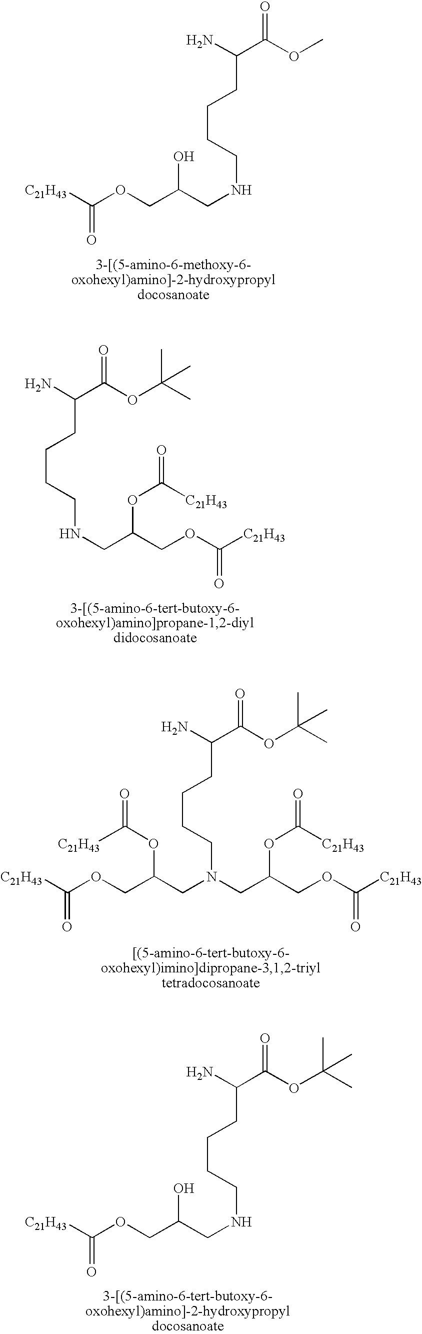

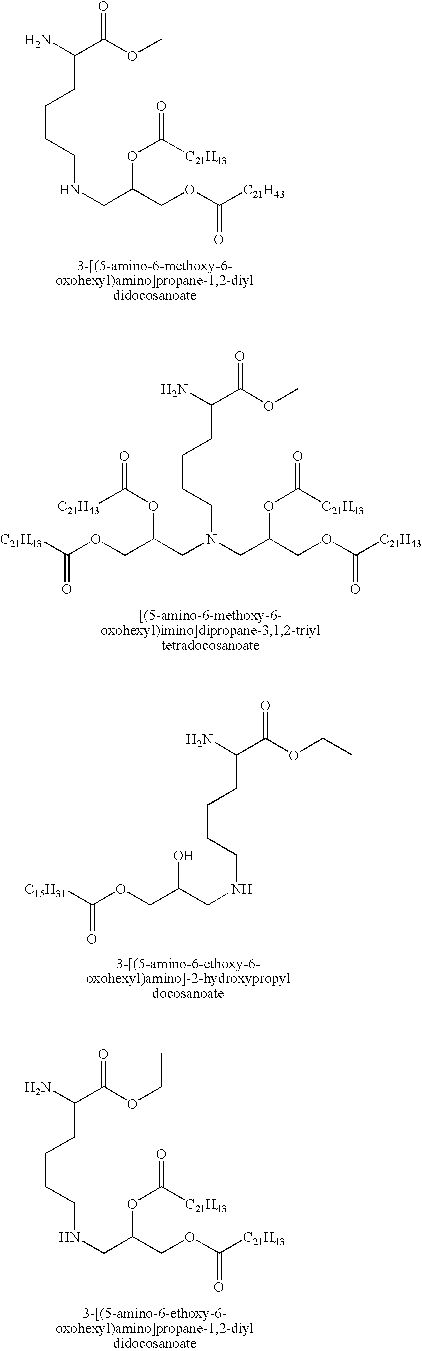

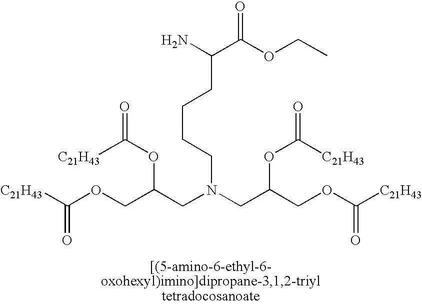

and the salts and/or solvates thereof.

For example, the present disclosure relates to a mixture of several entities chosen from lysine-derived compounds of formula (I) and the salts and/or solvates thereof, such as mono-, di-, tri- and tetrasubstituted esters.

For example, the mixture of the at least one entity chosen from lysine-derived compounds and the salts and/or solvates thereof can comprise from 0.5 to 15% by weight of monoesters, from 25 to 99.5% by weight of diesters, from 0.5 to 70% by weight of triesters and from 0.5 to 70% by weight of tetraesters, with respect to the total weight of the mixture.

According to at least one embodiment, the mixture of at least one entity chosen from lysine-derived compounds and the salts and/or solvates thereof comprises 6% by weight of 3-[(5-amino-6-methoxy-6-oxohexyl)(2,3-dihydroxypropyl)amino]-2-hydroxypropyl hexadecanoate, 55% by weight of [(5-amino-6-methoxy-6-oxohexyl)imino]bis-2-hydroxypropane-3,1-diyl dihexadecanoate and 36% by weight of a mixture of 3-{(5-amino-6-methoxy-6-oxohexyl)[2,3-bis(palmitoyloxy)propyl]amino}-2-hydroxypropyl palmitate and of [(5-amino-6-methoxy-6-oxohexyl)imino]dipropane-3,1,2-triyl tetrahexadecanoate.

Furthermore, the present disclosure relates to a cosmetic composition for conditioning keratinous fibers, for example human keratinous fibers, such as the hair, comprising at least one entity chosen from the specific lysine-derived compounds and the salts and/or solvates thereof as defined above.

Furthermore, another aspect of the present disclosure may be the use in cosmetics of at least one entity chosen from lysine-derived compound and the salts and/or solvates thereof as defined above.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLE 1

A cosmetic composition according to the disclosure was prepared from the ingredients shown below, the amounts of which are shown as percent by weight, with respect to the total weight of the composition.

| | Amount of |
|---|---|
| Composition | materials |
| [(5-Amino-6-methoxy-6-oxohexyl)imino]bis-2- | 3% as A.M. |
| hydroxypropane-3,1-diyl dihexadecanoate | |
| Water | q.s. for 100 |

A lock of bleached hair (SA20) weighing 0.5 g which was wetted beforehand was immersed in 15 g of the composition. The treatment was carried out at 30° C. for 15 minutes. The lock was subsequently rinsed and combed. It was observed that the wet lock exhibited a smooth feel, was soft and was easy to disentangle, while the control in water alone was rough and difficult to disentangle.

### EXAMPLE 2

A cosmetic composition according to the disclosure was prepared from the ingredients shown below, the amounts of which are shown as percent by weight, with respect to the total weight of the composition.

| | Amount of |
|---|---|
| Composition | materials |
| Mixture composed of 6% of 3-[(5-amino-6-methoxy- | 3% as A.M. |
| 6-oxohexyl)(2,3-dihydroxypropyl)amino]-2-hydroxypropyl | |
| hexadecanoate, of 55% of [(5-amino-6-methoxy-6- | |
| oxohexyl)imino]bis-2-hydroxypropane-3,1-diyl | |
| dihexadecanoate and of 36% of a mixture of 3-{(5-amino-6- | |
| methoxy-6-oxohexyl)[2,3-bis(palmitoyloxy)propyl]amino}- | |
| 2-hydroxypropyl palmitate and of [(5-amino-6-methoxy-6- | |
| oxohexyl)imino]dipropane-3,1,2-triyl tetrahexadecanoate | |
| Water | q.s. for 100 |

A lock of bleached hair (SA20) weighing 0.5 g which was wetted beforehand was immersed in 15 g of the composition. The treatment was carried out at 30° C. for 15 minutes. The lock was subsequently rinsed and combed. It was observed that the wet lock exhibited a smooth feel, was soft and was easy to disentangle, while the control in water alone was rough and difficult to disentangle.

### EXAMPLE 3

A cosmetic composition according to the disclosure was prepared from the ingredients shown below, the amounts of which are shown as percent by weight, with respect to the total weight of the composition.

| | Amount of |
|---|---|
| Composition | materials |
| Mixture composed of 0.7% of 3-[(5-amino-6- | 3% as A.M. |
| methoxy-6-oxohexyl)(2,3-dihydroxypropyl)amino]-2- | |
| hydroxypropyl hexadecanoate, of 26% of [(5-amino-6- | |
| methoxy-6-oxohexyl)imino]bis-2-hydroxypropane-3,1-diyl | |
| dihexadecanoate and of 70% of a mixture of 3-{(5-amino-6- | |
| methoxy-6-oxohexyl)[2,3-bis(palmitoyloxy)propyl]amino}- | |
| 2-hydroxypropyl palmitate and of [(5-amino-6-methoxy-6- | |
| oxohexyl)imino]dipropane-3,1,2-triyl tetrahexadecanoate | |
| Water | q.s. for 100 |

A lock of bleached hair (SA20) weighing 0.5 g which was wetted beforehand is immersed in 15 g of the composition. The treatment was carried out at 30° C. for 15 minutes. The lock was subsequently rinsed and combed. It was observed that the wet lock exhibited a smooth feel, was soft and was easy to disentangle, while the control in water alone was rough and difficult to disentangle.

What is claimed is:

1. A method for the conditioning of keratinous fibers comprising
applying, to the dry or wet fibers, a cosmetic composition comprising at least one entity chosen from lysine-derived compounds of formula (I), and the salts and/or solvates thereof: 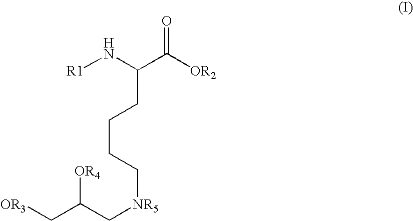
in which:
R₁ represents a hydrogen atom or a protective group;
R₂ represents a hydrogen atom, a linear C₁-C₆ alkyl radical or a branched C₃-C₆ alkyl radical;
R₃ and R₄ represent, independently of one another, a hydrogen atom, a saturated or unsaturated linear C₂-C₂₅ acyl group or a saturated or unsaturated branched C₄-C₂₅ acyl group;
R₅ represents a hydrogen atom or a CH₂CH(OR₆)CH₂OR₇ radical;
R₆ and R₇ represent, independently of one another, a hydrogen atom, a saturated or unsaturated linear C₃-C₂₅ acyl group or a saturated or unsaturated branched C₄-C₂₅ acyl group;
on the condition that R₁, R₂, R₃, R₄, R₅ and optionally R₆ and R₇ cannot simultaneously represent a hydrogen atom; and
optionally rinsing the cosmetic composition off after an optional leave-in time and/or after optionally drying the fibers.

2. A method according to claim 1, characterized in that the keratin fibers are human hair.

3. A method according to claim 1, characterized in that the R₁ protective group is chosen from a linear or branched C₂-C₆ acyl group; a linear or branched C₁-C₆ alkyl group; a C₁-C₆ carboxyalkoxy group; a (C₁-C₆)alkoxycarbonyl group; a linear or branched C₁-C₆ alkyl group interrupted by at least one entity chosen from nitrogen and carboxyl groups; a C₂-C₆ sulphonyl group; and an aryl group.

4. A method according to claim 3, characterized in that
the linear or branched C₂-C₆ acyl group is an acetyl group; and
the linear or branched C₁-C₆ alkyl group is a methyl or ethyl group.

5. A method according to claim 1, characterized in that R₁ represents a hydrogen atom; R₂ represents a linear C₁-C₄ alkyl radical, or a branched C₃-C₄ alkyl radical; R₃ and R₄ represent, independently of one another, a hydrogen atom or a saturated or unsaturated and linear or branched C₁₂-C₂₂ acyl group; R₅ represents a hydrogen atom or a CH₂CH(OR₆)CH₂OR₇ group; R₆ and R₇ represent, independently of one another, a hydrogen atom or a saturated or unsaturated and linear or branched C₁₂-C₂₂ acyl group; and R₁, R₂, R₃, R₄, R₅ and optionally R₆ and R₇ cannot simultaneously represent a hydrogen atom.

6. A method according to claim 5, characterized in that
the R₂ linear C₁-C₄ alkyl radical is a methyl or ethyl group;
the R₂ branched C₃-C₄ alkyl radical is a tert-butyl group;
the R₃ and R₄ are independently of one another, a C₁₆-C₂₂, acyl group; and
the R₆ and R₇ are, independently of one another, a C₁₆-C₂₂, acyl group.

7. A method according to claim 1, characterized in that at least one entity chosen from lysine-derived compounds of formula (I), is chosen from:
3-[(5-amino-6-tert-butoxy-6-oxohexyl)(2,3-dihydroxypropyl)amino]-2-hydroxypropyl hexadecanoate;
3-[(5-amino-6-tert-butoxy-6-oxohexyl)(2,3-dihydroxypropyl)amino]-2-hydroxypropyl dodecanoate;
3-[(5-amino-6-methoxy-6-oxohexyl)(2,3-dihydroxypropyl)amino]-2-hydroxypropyl hexadecanoate;
3-[(5-amino-6-methoxy-6-oxohexyl)(2,3-dihydroxypropyl)amino]-2-hydroxypropyl dodecanoate;
3-[(5-amino-6-ethoxy-6-oxohexyl)(2,3-dihydroxypropyl)amino]-2-hydroxypropyl hexadecanoate;
3-[(5-amino-6-ethoxy-6-oxohexyl)(2,3-dihydroxypropyl)amino]-2-hydroxypropyl dodecanoate;
[(5-amino-6-tert-butoxy-6-oxohexyl)imino]bis-2-hydroxypropane-3,1-diyl dihexadecanoate;
[(5-amino-6-tert-butoxy-6-oxohexyl)imino]bis-2-hydroxypropane-3,1-diyl didodecanoate;
[(5-amino-6-methoxy-6-oxohexyl)imino]bis-2-hydroxypropane-3,1-diyl dihexadecanoate;
[(5-amino-6-methoxy-6-oxohexyl)imino]bis-2-hydroxypropane-3,1-diyl didodecanoate;
[(5-amino-6-ethoxy-6-oxohexyl)imino]bis-2-hydroxypropane-3,1-diyl dihexadecanoate;
[(5-amino-6-ethoxy-6-oxohexyl)imino]bis-2-hydroxypropane-3,1-diyl didodecanoate;
3-{(5-amino-6-tert-butoxy-6-oxohexyl)[2,3-bis(palmitoyloxy)propyl]amino}-2-hydroxypropyl palmitate;
3-{(5-amino-6-tert-butoxy-6-oxohexyl)[2,3-bis(dodecanoyloxy)propyl]amino}-2-hydroxypropyl laurate;
3-{(5-amino-6-methoxy-6-oxohexyl)[2,3-bis(palmitoyloxy)propyl]amino}-2-hydroxypropyl palmitate;
3-{(5-amino-6-methoxy-6-oxohexyl)[2,3-bis(dodecanoyloxy)propyl]amino}-2-hydroxypropyl laurate;
3-{(5-amino-6-ethoxy-6-oxohexyl)[2,3-bis(palmitoyloxy)propyl]amino}-2-hydroxypropyl palmitate;
3-{(5-amino-6-ethoxy-6-oxohexyl)[2,3-bis(dodecanoyloxy)propyl]amino}-2-hydroxypropyl laurate;
[(5-amino-6-tert-butoxy-6-oxohexyl)imino]dipropane-3,1,2-triyl tetrahexadecanoate;
[(5-amino-6-tent-butoxy-6-oxohexyl)imino]dipropane-3,1,2-triyl tetradodecanoate;
[(5-amino-6-methoxy-6-oxohexyl)imino]dipropane-3,1,2-triyl tetrahexadecanoate;
[(5-amino-6-methoxy-6-oxohexyl)imino]dipropane-3,1,2-triyl tetradodecanoate;
[(5-amino-6-ethoxy-6-oxohexyl)imino]dipropane-3,1,2-triyl tetrahexadecanoate;
[(5-amino-6-ethoxy-6-oxohexyl)imino]dipropane-3,1,2-triyl tetradodecanoate;
3-[(5-amino-6-tert-butoxy-6-oxohexyl)amino]-2-hydroxypropyl palmitate;
3-[(5-amino-6-tert-butoxy-6-oxohexyl)amino]-2-hydroxypropyl laurate;
3-[(5-amino-6-methoxy-6-oxohexyl)amino]-2-hydroxypropyl palmitate;
3-[(5-amino-6-methoxy-6-oxohexyl)amino]-2-hydroxypropyl laurate;
3-[(5-amino-6-ethoxy-6-oxohexyl)amino]-2-hydroxypropyl palmitate;
3-[(5-amino-6-ethoxy-6-oxohexyl)amino]-2-hydroxypropyl laurate;
3-[(5-amino-6-tert-butoxy-6-oxohexyl)amino]propane-1,2-diyl dihexadecanoate;
3-[(5-amino-6-tert-butoxy-6-oxohexyl)amino]propane-1,2-diyl didodecanoate;
3-[(5-amino-6-methoxy-6-oxohexyl)amino]propane-1,2-diyl dihexadecanoate;
3-[(5-amino-6-methoxy-6-oxohexyl)amino]propane-1,2-diyl didodecanoate;
3-[(5-amino-6-ethoxy-6-oxohexyl)amino]propane-1,2-diyl dihexadecanoate;
3-[(5-amino-6-ethoxy-6-oxohexyl)amino]propane-1,2-diyl didodecanoate;
3-[(5-amino-6-tert-butoxy-6-oxohexyl)(2,3-dihydroxypropyl)amino]-2-hydroxypropyl docosanoate;
3-[(5-amino-6-methoxy-6-oxohexyl)(2,3-dihydroxypropyl)amino]-2-hydroxypropyl docosanoate;
3-[(5-amino-6-ethoxy-6-oxohexyl)(2,3-dihydroxypropyl)amino]-2-hydroxypropyl docosanoate;
[(5-amino-6-tert-butoxy-6-oxohexyl)imino]bis-2-hydroxypropane-3,1-diyl didocosanoate;
3-{(5-amino-6-tert-butoxy-6-oxohexyl)[2,3-bis(docosanoyloxy)propyl]amino}-2-hydroxypropyl docosanoate;
[(5-amino-6-methoxy-6-oxohexyl)imino]bis-2-hydroxypropane-3,1-diyl didocosanoate;
3-{(5-amino-6-methoxy-6-oxohexyl)[2,3-bis(docosanoyloxy)propyl]amino}-2-hydroxypropyl docosanoate;
[(5-amino-6-ethoxy-6-oxohexyl)imino]bis-2-hydroxypropane-3,1-diyl didocosanoate;
3-{(5-amino-6-ethoxy-6-oxohexyl)[2,3-bis(docosanoyloxy)propyl]amino}-2-hydroxypropyl docosanoate;
3-[(5-amino-6-methoxy-6-oxohexyl)amino]-2-hydroxypropyl docosanoate;
3-[(5-amino-6-tert-butoxy-6-oxohexyl)amino]propane-1,2-diyl didocosanoate;
[(5-amino-6-tert-butoxy-6-oxohexyl)imino]dipropane-3,1,2-triyl tetradocosanoate;
3-[(5-amino-6-tert-butoxy-6-oxohexyl)amino]-2-hydroxypropyl docosanoate;
3-[(5-amino-6-methoxy-6-oxohexyl)amino]propane-1,2-diyl didocosanoate;
[(5-amino-6-methoxy-6-oxohexyl)imino]dipropane-3,1,2-triyl tetradocosanoate;
3-[(5-amino-6-ethoxy-6-oxohexyl)amino]-2-hydroxypropyl docosanoate;
3-[(5-amino-6-ethoxy-6-oxohexyl)amino]propane-1,2-diyl didocosanoate;
[(5-amino-6-ethyl-6-oxohexyl)imino]dipropane-3,1,2-triyl tetradocosanoate
and the salts and/or solvates thereof.

8. A method according to claim 1, characterized in that the salts are inorganic salts chosen from hydrohalides, carbonates, hydrogencarbonates, sulphates, hydrogenphosphates, and phosphates.

9. A method according to claim 1, characterized in that the salts are organic salts chosen from salts of organic acids.

10. A method according to claim 9, characterized in that the salts of organic acids are chosen from citrates, lactates, glycolates, gluconates, acetates, propionates, fumarates, oxalates, and tartrates.

11. A method according to claim 1, characterized in that the solvates are chosen from hydrates, alcoholates, and hydroalcoholates.

12. A cosmetic composition for conditioning keratinous fibers comprising, in a cosmetically acceptable medium, at least one entity chosen from lysine-derived compounds of formula (I) and the salts and/or solvates thereof: 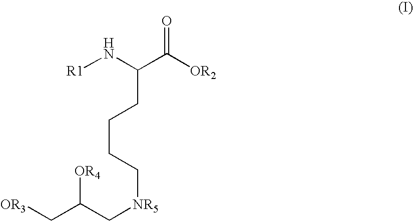
in which:
R₁ represents a hydrogen atom or a protective group;
R₂ represents a hydrogen atom, a linear C₁-C₆ alkyl radical or a branched C₃-C₆ alkyl radical;
R₃ and R₄ represent, independently of one another, a hydrogen atom, a saturated or unsaturated linear C₂-C₂₅ acyl group or a saturated or unsaturated branched C₄-C₂₅ acyl group;
R₅ represents a hydrogen atom or a CH₂CH(OR₆)CH₂OR₇ radical;
R₆ and R₇ represent, independently of one another, a hydrogen atom, a saturated or unsaturated linear C₃-C₂₅ acyl group or a saturated or unsaturated branched C₄-C₂₅ acyl group;
on the condition that R₁, R₂, R₃, R₄, R₅ and optionally R₆ and R₇ cannot simultaneously represent a hydrogen atom; and
at least one cosmetic adjuvant.

13. A cosmetic composition according to claim 12, characterized in that the keratinous fiber is hair.

14. A cosmetic composition according to claim 12, characterized in that the at least one entity chosen from lysine-derived compounds of formula (I) is present in an amount ranging from 0.01 to 50% by weight, with respect to the total weight of the composition.

15. A cosmetic composition according to claim 14, characterized in that the at least one compound of formula (I) is present in an amount ranging from 1.5 and 10% by weight, with respect to the total weight of the composition.

16. A cosmetic composition according to claim 12, characterized in that the at least one cosmetic adjuvant is chosen from anionic surfactants, cationic surfactants other than the entities of formula (I), amphoteric surfactants, and nonionic surfactants; cationic polymers, anionic polymers, amphoteric polymers, zwitterionic polymers and nonionic polymers; and silicones.

17. A cosmetic composition according to claim 16, characterized in that the at least one cosmetic adjuvant is a cationic surfactant chosen from optionally polyoxyalkylenated primary, secondary or tertiary fatty amine salts; quaternary ammonium salts; imidazoline derivatives; and amine oxides having a cationic nature.

18. A cosmetic composition according to claim 17, characterized in that said the at least one quaternary ammonium salt is chosen from tetraalkylammonium, alkylamidoalkyltrialkylammonium, trialkylbenzylammonium, trialkylhydroxyalkylammonium, and alkylpyridinium chlorides or bromides.

19. A cosmetic composition according to claim 16, characterized in that the at least one cosmetic adjuvant is chosen from cationic polymers.

20. A cosmetic composition according to claim 19, characterized in that the at least one cationic polymer is chosen from quaternary cellulose ether derivatives; quaternized guar gum; cationic cyclopolymers; and noncrosslinked and crosslinked polymers of methacryloyloxy(C₁-C₄)alkyltri(C₁-C₄)alkylammonium salts.

21. A cosmetic composition according to claim 20, characterized in that the at least one cationic cyclopolymer is chosen from dimethyldiallylammonium chloride homopolymers or copolymers, and quaternary copolymers of vinylpyrrolidone and of vinylimidazole.

22. A cosmetic composition according to claim 20, characterized in that the at least one cationic polymer is chosen from dimethyldiallylammonium chloride homopolymers or copolymers.

23. A cosmetic composition according to claim 16, characterized in that the at least one cosmetic adjuvant is chosen from silicones.

24. A cosmetic composition according to claim 12, characterized in that the at least one cosmetic adjuvant is present in an amount ranging from 0.01 to 50% by weight, with respect to the total weight of the composition.

25. A cosmetic composition according to claim 24, characterized in that the cosmetic adjuvant is present at a concentration ranging from 0.1 to 30% by weight, with respect to the total weight of the composition.

26. A cosmetic composition according to claim 12, characterized in that the cosmetically acceptable medium is an aqueous, alcoholic, or aqueous/alcoholic medium.

27. A cosmetic composition according to claim 26, characterized in that the aqueous/alcoholic medium comprises at least one alcohol chosen from C₁-C₄ alcohols, polyols, and polyol monoethers.

28. A cosmetic composition according to claim 27, characterized in that the at least one alcohol is ethanol.

29. A cosmetic composition according to claim 12, characterized in that the composition is a conditioner or a shampoo.

30. A method for making a cosmetic composition for keratinous fibers, comprising combining, in a cosmetically acceptable medium,
at least one entity chosen from lysine-derived compounds of formula (I) and the salts and/or solvates thereof: 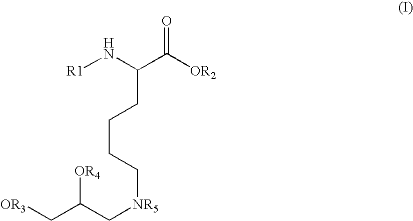
in which:
R₁ represents a hydrogen atom or a protective group;
R₂ represents a hydrogen atom, a linear C₁-C₆ alkyl radical or a branched C₃-C₆ alkyl radical;
R₃ and R₄ represent, independently of one another, a hydrogen atom, a saturated or unsaturated linear C₂-C₂₅ acyl group or a saturated or unsaturated branched C₄-C₂₅ acyl group;
R₅ represents a hydrogen atom or a CH₂CH(OR₆)CH₂OR₇ radical;
R₆ and R₇ represent, independently of one another, a hydrogen atom, a saturated or unsaturated linear C₃-C₂₅ acyl group or a saturated or unsaturated branched C₄-C₂₅ acyl group;
on the condition that R₁, R₂, R₃, R₄, R₅ and optionally R₆ and R₇ cannot simultaneously represent a hydrogen atom; and
at least one adjuvant.

31. At least one entity chosen from lysine-derived compounds chosen from:
3-[(5-amino-6-tert-butoxy-6-oxohexyl)(2,3-dihydroxypropyl)amino]-2-hydroxypropyl hexadecanoate;
3-[(5-amino-6-tert-butoxy-6-oxohexyl)(2,3-dihydroxypropyl)amino]-2-hydroxypropyl dodecanoate;
3-[(5-amino-6-methoxy-6-oxohexyl)(2,3-dihydroxypropyl)amino]-2-hydroxypropyl hexadecanoate;
3-[(5-amino-6-methoxy-6-oxohexyl)(2,3-dihydroxypropyl)amino]-2-hydroxypropyl dodecanoate;
3-[(5-amino-6-ethoxy-6-oxohexyl)(2,3-dihydroxypropyl)amino]-2-hydroxypropyl hexadecanoate;
3-[(5-amino-6-ethoxy-6-oxohexyl)(2,3-dihydroxypropyl)amino]-2-hydroxypropyl dodecanoate;
[(5-amino-6-tert-butoxy-6-oxohexyl)imino]bis-2-hydroxypropane-3,1-diyl dihexadecanoate;
[(5-amino-6-tert-butoxy-6-oxohexyl)imino]bis-2-hydroxypropane-3,1-diyl didodecanoate;
[(5-amino-6-methoxy-6-oxohexyl)imino]bis-2-hydroxypropane-3,1-diyl dihexadecanoate;
[(5-amino-6-ethoxy-6-oxohexyl)imino]bis-2-hydroxypropane-3,1-diyl dihexadecanoate;
[(5-amino-6-ethoxy-6-oxohexyl)imino]bis-2-hydroxypropane-3,1-diyl didodecanoate;
3-{(5-amino-6-tert-butoxy-6-oxohexyl)[2,3-bis(palmitoyloxy)propyl]amino}-2-hydroxypropyl palmitate;
3-{(5-amino-6-tert-butoxy-6-oxohexyl)[2,3-bis(dodecanoyloxy)propyl]amino}-2-hydroxypropyl laurate;
3-{(5-amino-6-methoxy-6-oxohexyl)[2,3-bis(palmitoyloxy)propyl]amino}-2-hydroxypropyl palmitate;
3-{(5-amino-6-methoxy-6-oxohexyl)[2,3-bis(dodecanoyloxy)propyl]amino}-2-hydroxypropyl laurate;
3-{(5-amino-6-ethoxy-6-oxohexyl)[2,3-bis(palmitoyloxy)propyl]amino}-2-hydroxypropyl palmitate;
3-{(5-amino-6-ethoxy-6-oxohexyl)[2,3-bis(dodecanoyloxy)propyl]amino}-2-hydroxypropyl laurate;
[(5-amino-6-tert-butoxy-6-oxohexyl)imino]dipropane-3,1,2-triyl tetrahexadecanoate;
[(5-amino-6-tert-butoxy-6-oxohexyl)imino]dipropane-3,1,2-triyl tetradodecanoate;
[(5-amino-6-methoxy-6-oxohexyl)imino]dipropane-3,1,2-triyl tetrahexadecanoate;
[(5-amino-6-methoxy-6-oxohexyl)imino]dipropane-3,1,2-triyl tetradodecanoate;
[(5-amino-6-ethoxy-6-oxohexyl)imino]dipropane-3,1,2-triyl tetrahexadecanoate;
[(5-amino-6-ethoxy-6-oxohexyl)imino]dipropane-3,1,2-triyl tetradodecanoate;
3-[(5-amino-6-tert-butoxy-6-oxohexyl)amino]-2-hydroxypropyl palmitate;
3-[(5-amino-6-tert-butoxy-6-oxohexyl)amino]-2-hydroxypropyl laurate;
3-[(5-amino-6-methoxy-6-oxohexyl)amino]-2-hydroxypropyl palmitate;
3-[(5-amino-6-methoxy-6-oxohexyl)amino]-2-hydroxypropyl laurate;
3-[(5-amino-6-ethoxy-6-oxohexyl)amino]-2-hydroxypropyl palmitate;
3-[(5-amino-6-ethoxy-6-oxohexyl)amino]-2-hydroxypropyl laurate;
3-[(5-amino-6-tert-butoxy-6-oxohexyl)amino]propane-1,2-diyl dihexadecanoate;
3-[(5-amino-6-tert-butoxy-6-oxohexyl)amino]propane-1,2-diyl didodecanoate;
3-[(5-amino-6-methoxy-6-oxohexyl)amino]propane-1,2-diyl dihexadecanoate;
3-[(5-amino-6-methoxy-6-oxohexyl)amino]propane-1,2-diyl didodecanoate;
3-[(5-amino-6-ethoxy-6-oxohexyl)amino]propane-1,2-diyl dihexadecanoate;
3-[(5-amino-6-ethoxy-6-oxohexyl)amino]propane-1,2-diyl didodecanoate;
3-[(5-amino-6-tert-butoxy-6-oxohexyl)(2,3-dihydroxypropyl)amino]-2-hydroxypropyl docosanoate;
3-[(5-amino-6-methoxy-6-oxohexyl)(2,3-dihydroxypropyl)amino]-2-hydroxypropyl docosanoate;
3-[(5-amino-6-ethoxy-6-oxohexyl)(2,3-dihydroxypropyl)amino]-2-hydroxypropyl docosanoate;
[(5-amino-6-tert-butoxy-6-oxohexyl)imino]bis-2-hydroxypropane-3,1-diyl didocosanoate;
3-{(5-amino-6-tert-butoxy-6-oxohexyl)[2,3-bis(docosanoyloxy)propyl]amino}-2-hydroxypropyl docosanoate;
[(5-amino-6-methoxy-6-oxohexyl)imino]bis-2-hydroxypropane-3,1-diyl didocosanoate;
3-{(5-amino-6-methoxy-6-oxohexyl)[2,3-bis(docosanoyloxy)propyl]amino}-2-hydroxypropyl docosanoate;
[(5-amino-6-ethoxy-6-oxohexyl)imino]bis-2-hydroxypropane-3,1-diyl didocosanoate;
3-{(5-amino-6-ethoxy-6-oxohexyl)[2,3-bis(docosanoyloxy)propyl]amino}-2-hydroxypropyl docosanoate;
3-[(5-amino-6-methoxy-6-oxohexyl)amino]-2-hydroxypropyl docosanoate;
3-[(5-amino-6-tert-butoxy-6-oxohexyl)amino]propane-1,2-diyl didocosanoate;
[(5-amino-6-tert-butoxy-6-oxohexyl)imino]dipropane-3,1,2-triyl tetradocosanoate;
3-[(5-amino-6-tert-butoxy-6-oxohexyl)amino]-2-hydroxypropyl docosanoate;
3-[(5-amino-6-methoxy-6-oxohexyl)amino]propane-1,2-diyl didocosanoate;
[(5-amino-6-methoxy-6-oxohexyl)imino]dipropane-3,1,2-triyl tetradocosanoate;
3-[(5-amino-6-ethoxy-6-oxohexyl)amino]-2-hydroxypropyl docosanoate;
3-[(5-amino-6-ethoxy-6-oxohexyl)amino]propane-1,2-diyl didocosanoate;
[(5-amino-6-ethyl-6-oxohexyl)imino]dipropane-3,1,2-triyl tetradocosanoate
and the salts and/or solvates thereof.

32. At least one entity chosen from lysine-derived compounds of formula (I) and the salts and/or solvates thereof: 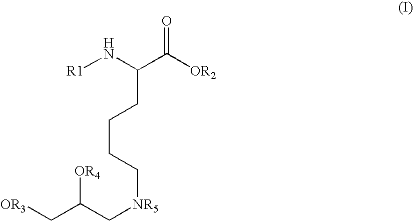
in which:
R₁ represents a hydrogen atom or a protective group;
R₂ represents a hydrogen atom, a linear C₁-C₆ alkyl radical or a branched C₃-C₆ alkyl radical;
R₃ and R₄ represent, independently of one another, a hydrogen atom, a saturated or unsaturated linear C₂-C₂₅ acyl group or a saturated or unsaturated branched C₄-C₂₅ acyl group;
R₅ represents a hydrogen atom or a CH₂CH(OR₆)CH₂OR₇ radical;
R₆ and R₇ represent, independently of one another, a hydrogen atom, a saturated or unsaturated linear C₃-C₂₅ acyl group or a saturated or unsaturated branched C₄-C₂₅ acyl group;
on the condition that R₁, R₂, R₃, R₄, R₅ and optionally R₆ and R₇ cannot simultaneously represent a hydrogen atom;
comprising from 0.5 to 15% by weight of monoesters, from 25 to 99.5% by weight of diesters, from 0.5 to 70% by weight of triesters and from 0.5 to 70% by weight of tetraesters, with respect to the total weight of the mixture.

33. A mixture according to claim 32, characterized in that it comprises 6% by weight of 3-[(5-amino-6-methoxy-6-oxohexyl)(2,3-dihydroxypropyl)amino]-2-hydroxypropyl hexadecanoate, 55% by weight of [(5-amino-6-methoxy-6-oxohexyl)imino]bis-2-hydroxypropane-3,1-diyl dihexadecanoate and 36% by weight of a mixture of 3-{(5-amino-6-methoxy-6-oxohexyl)[2,3-bis(palmitoyloxy)propyl]amino}-2-hydroxypropyl palmitate and of [(5-amino-6-methoxy-6-oxohexyl)imino]dipropane-3,1,2-triyl tetrahexadecanoate.
